(19) United States
(12) Patent Application Publication
(10) Pub. No.: US 2020/0360322 A1
(43) Pub. Date: Nov. 19, 2020

(54) FORMULATIONS AND DOSAGE FORMS FOR ENHANCING PERFORMANCE OR RECOVERY FROM STRESS

(71) Applicant: ACTIVATE BIO PHYSICS CORP., Vancouver (CA)

(72) Inventors: Evgeny I. Maevsky, Pushchino (RU); Mikhail V. Kozhurin, Saint Petersburg (RU); Marina E. Maevskaya, Moscow (RU)

(21) Appl. No.: 16/763,532

(22) PCT Filed: Nov. 15, 2018
(86) PCT No.: PCT/US2018/061371

§ 371 Date: May 12, 2020

Related U.S. Application Data
(60) Provisional application No. 62/678,124, filed on May 30, 2018.
(60) Provisional application No. 62/586,665, filed on Nov. 15, 2017.

(51) Int. Cl. A61K 31/194; A61K 47/38; A61K 47/02; A61K 47/12; A61K 9/50; A61P 9/00; A61K 33/42; A61K 31/205; A61K 9/20; A61K 9/28

(52) U.S. Cl. CPC A61K 31/194; A61K 47/38; A61K 47/02; A61K 47/12; A61K 9/5047; A61P 9/00; A61K 9/0053; A61K 31/205; A61K 9/2009; A61K 9/2013; A61K 9/2054; A61K 9/28; A61K 33/42

(57) ABSTRACT

The present disclosure provides formulations and dosages forms thereof comprising ammonium succinate for enhancing physical performance and recovery after physical performance.

### BACKGROUND

### Technical Field

This disclosure relates to dosage forms for enhancing performance and accelerating recovery, and uses thereof.

### Description of the Related Art

Hypoxic/ischemic episodes and events are known to be a natural way of mobilizing the body's adaptive responses [1, 2, 3]. In general, preconditioning means that recurrent brief ischemic or hypoxic stresses or induced episodes of intermittent hypoxia that simulate them significantly increase the tolerance of the body and isolated organs to subsequent hypoxic or ischemic events as well as to reoxygenation (associated with free radical generation and increased stress on the body's antioxidant systems). Hypoxic ischemic pre-conditioning (HIPC) lies at the core of exercises aimed at the preparation for mental, psycho-emotional and physical activities of extreme intensity. This concept is used for creating methods and remedies for the physiotherapeutic and pharmacological prevention and treatment of illnesses, for which hypoxia or ischemia, as well as stress on antioxidant systems, plays a leading pathogenic role. Generally, anti-hypoxic and anti-ischemic pharmacological agents decrease the activity of tissues and organs. Thus, energy expenditure and oxygen demand are reduced [2]. With the help of high doses of exogenous antioxidants, the function of natural antioxidant systems is not simply supplemented, but essentially replaced. Instead of conditioning, excessive antioxidant replacement therapy causes a weakening of endogenous antioxidant systems and the loss of free radical regulation, which contributes to carcinogenesis and aging [4, 5, 6].

Agents improving the function of the existing vascular network are often used concurrently. Among the pharmacological agents used are adenosine; bradykinin; opiates; various types of anesthetics; β-blockers; inhibitors of fatty acid mobilization and oxidation and of carnitine synthesis; Na+/H+ exchange inhibitors; hypoglycemic anti-diabetics; and massive doses of antioxidants. Essentially, polyfunctional replacement therapy takes place, turning off natural adaptive processes. It is worth noting that the latter is appropriate when endogenous defense systems are threatened or exhausted. Examples include ischemic damage that has already occurred, or a severe and prolonged pathological process, especially when the body is aging and its endogenous adaptive systems, including its antioxidant systems, are being depleted.

However, given actual extreme stresses, such pharmacological interventions lead to stress avoidance rather than the activation of adaptive systems and recovery, which is not acceptable in a number of cases. For example, in sports, where HIPC is achieved through a specific training program, HIPC is aimed at the completely different purpose of enhancing performance and restoring mobilization [1, 3].

Therefore, there is a need for a different pharmacological approach, using compositions that increase tolerance to ischemia and hypoxia not by reducing capacity or energy expenditure, but rather by activating natural adaptive processes and increasing the efficiency of the body. Presently disclosed embodiments address this need and provide other related advantages.

### BRIEF SUMMARY

The present disclosure provides formulations and dosage forms that may be used for enhancing physical performance and recovery from stress.

In one aspect, the present disclosure provides a dosage form for oral administration comprising a formulation comprising: about 24%-36% by weight ammonium succinate; about 4%-16% by weight sodium succinate; about 4%-12% by weight succinic acid; about 8%-16% by weight potassium fumarate or sodium fumarate; about 2%-28% by weight ammonium phosphate; and about 8%-16% by weight L-carnitine fumarate or a mixture of L-carnitine and sodium fumarate at a 1:1 ratio.

In another aspect, the present disclosure provides a dosage form for oral administration comprising a formulation comprising: about 54%-76% by weight ammonium succinate; about 6%-14% by weight sodium succinate; and about 6%-14% by weight ascorbic acid.

In yet another aspect, the present disclosure provides a dosage form for oral administration comprising a formulation comprising: about 36%-48% by weight ammonium succinate; about 7%-14% by weight sodium succinate; about 4%-9% by weight succinic acid; about 4%-7% by weight potassium fumarate or sodium fumarate; about 2%-4% by weight ammonium phosphate; about 8%-14% by weight L-carnitine fumarate or a mixture of L-carnitine and sodium fumarate at a 1:1 ratio; about 1%-3% by weight antioxidant; about 4%-10% by weight dihydroquercetin; about 4%-10% by weight α-tocopherol; and about 4%-14% by weight ascorbic acid. In certain embodiments, the antioxidant is lycopene or curcumin.

In another aspect, the present disclosure provides methods of enhancing physical performance or recovery following physical performance in a subject comprising orally administering an effective amount of the dosage forms provided herein to a subject. In certain embodiments, the dosage form is administered to the subject before or after exercise.

### BRIEF DESCRIPTION OF THE SEVERAL VIEWS OF THE DRAWINGS

FIG. 1 is a schematic showing concentration (or quantitative) gradient scheme of substances introduced through the gastro-intestinal tract as the substances move through the body.

FIG. 2 is a diagram depicting anaerobic intracellular energy metabolism as a source of extracellular metabolites responsible for the formation of an adaptive signal for ischemia. The substances that escape into the bloodstream are underlined.

FIG. 3 is a diagram depicting aerobic metabolism of succinate and fumarate in mitochondria.

FIG. 4 is a diagram depicting the reduction of fumarate to succinate under anaerobic conditions.

FIGS. 5A-5C are a bar graphs depicting changes in pH (FIG. 5A), buffer base shift (BBS) (FIG. 5B) and lactate/pyruvate ratio (FIG. 5C) in blood in rats following a swimming test. The rats were treated before the test with a control solution, an actoprotector [7], or Formulation E.

FIGS. 6A-6D are bar graphs depicting the effect of Formulation H.

FIGS. 6A and 6B show standard physical load and the change in blood pH following exercise for athletes treated before exercise with placebo or with Formulation H. FIG. 6C shows the work capacity of the athletes following a prior administration of either placebo or Formulation H. FIG. 6D shows the change in blood pH following this second round of exercise.

FIGS. 7A-7B are bar graphs depicting the degree of restoration of working capacity in athletes after a short rest. FIG. 7A shows the results for athletes administered a placebo. FIG. 7B shows the results for athletes administered Formulation H.

FIG. 8 is a set of bar graphs depicting a comparison of capacity to perform repeated loads after a break. The average changes in pH after loads are shown on the top axis and the power of the work performed is shown on the bottom axis. Statistically significant differences were observed: *p<0.05; **p<0.01; #p<0.05.

FIGS. 9A-9B show exemplary dosage regimens for formulations of the present disclosure. FIG. 9A shows exemplary dosage regimens for the “Active Formulation.” FIG. 9B shows exemplary dosage regimens for the “Pro-Sport Formulation.”

### DETAILED DESCRIPTION

The present disclosure provides a pharmacometabolic approach for enhancing physical performance and recovery following physical performance using formulations comprising metabolic substances that increase tolerance to ischemia and hypoxia, not so much by reducing capacity or energy expenditure, but rather by activating and triggering natural adaptive processes and increasing the efficiency of the entire body. In particular, oxygen is used more efficiently and the share of the aerobic energy supply of work is increased correspondingly. Increasing the efficiency of oxygen utilization reduces stress on the heart. At the root of this approach is the usage of metabolites released into the bloodstream during short-term hypoxia and/or ischemia. The metabolites in the formulations provided in the present disclosure hold key positions in biochemical transformations and possess pronounced signaling activity. These metabolites specifically interact with cellular receptors and have an effect on the expression of genes responsible for triggering adaptive anti-ischemic processes.

The formulations and dosages forms of the present disclosure reproduce the main elements of HIPC, i.e., the effect is similar to what occurs in nature during short recurrent pulses of hypoxia and/or ischemia. The formulations and dosage forms of the present disclosure sustain the actions of natural factors but do not replace them. As a result, the formulations and dosage forms of the present disclosure reduce fatigability, enhances performance, increases the rate of recovery after intense stresses, and aids in the preservation of functional activity in old age.

The formulations and dosage forms of the present disclosure includes catabolic intermediates, inorganic acid salts, as well as small quantities of antioxidants. In certain embodiments, the formulations and dosage forms comprise succinic, fumaric and pyruvic acids in the forms of sodium, magnesium, potassium, calcium and ammonium salts, L-carnitine fumarate, ammonium phosphate, sodium bicarbonate, ascorbic acid, dihydroquercetin, lycopene, and Vitamin E. In certain embodiments, the formulations and dosage forms further comprise inorganic salts to maintain an acid-base balance.

An actoprotector whose active substance is the ammonium salt of succinic acid (ammonium succinate) has been described (Russian Patent No. 2121836 [7]). Despite its actoprotective activity [8, 9, 10], the agent of the Russian Patent No. 2121836 has not found broad therapeutic utility because of unpredictable effects and because of its noticeable local irritant effect. As such, instead of an effect of increased activity, a number of people always experience an inhibitory, sedative and even hypnotic effect, while some other people always experience this effect only when fatigued. In the case of this inhibitory effect, heaviness in the hands and legs may be felt; occasionally a depressive state may develop. These are reproducible, not random, responses.

It is worth noting that, even in the 19^{th} century, a component like ammonium succinate was included in the composition of pharmaceutical sedatives such as liquor antispasticus and mixtura tonico nervina [11].

The local irritant effect of ammonium succinate itself has been found to be more pronounced when the dose is increased or when it is taken repeatedly. The local irritant effect can sometimes occur even after a single dose in users with histories of gastritis or peptic ulcer disease of the stomach and duodenum.

Ammonium succinate has been administered as a geroprotector as well [12]. Injecting old male rats with ammonium succinate for several days leads to an increase in the content of energy-rich ATP and ADP compounds in the liver to a level characteristic of young animals.

Russian Patent No. 2108095 presents ammonium succinate as a “medicinal agent for the treatment of cerebral ischemia, aiding in the correction of disruptions in blood supply and cerebral metabolism” [13].

The data produced by A. M. Gurvich et al. raise some doubts with respect to ammonium succinate's protective anti-ischemic effect on the brain [14, 15]. According to this research, an abdominal injection of ammonium succinate or sodium succinate after a 10-minute stop of blood flow in an animal not only fails to improve, but can aggravate, the condition of the central nervous system. On the contrary, oral administration of succinic acid improves the central nervous system's recovery [16].

The commercially available dietary supplement Yantarin-Sport [17] contains 0.225 g of sodium succinate, 0.33 g of glutamic acid, 0.15 g of L-arginine, 0.015 g of riboflavin and 0.03 g of pyridoxine. Yantarin-Sport is offered as a “natural energy drink” without caffeine. Yantarin-Sport advertisements state that this dietary supplement enhances performance and accelerates recovery in actively working people. However, despite its complex composition, Yantarin-Sport has been found to be less effective that the U.S. Pat. No. 2,121,836 actoprotector with a simpler composition. Yantarin-Sport distributors make unsubstantiated claims about the dietary supplement's participation in anabolism and catabolism based on data from a biochemistry textbook. A test has shown that the substrate doses in Yantarin-Sport are so small that their contribution to energetics cannot reach 1% of the body's energy needs, even in a resting state. Under stress, energy needs increase manyfold [1]. Yantarin-Sport's low effectiveness can be due to a slow release of its components from the gastrointestinal tract into the bloodstream. For example, glutamic acid does not dissolve well in water and therefore its absorption occurs very slowly. As a result, a significant portion of the Yantarin-Sport dietary supplement's components is eliminated in the gastrointestinal tract by mucosal cells and microbiota, and their impact on the body's anabolism and catabolism is weak. There are other reasons as well. The amount of L-arginine in this dietary supplement's composition is 10 times lower than the dose required to enhance protein synthesis and activate production of the NO vasoactive messenger. On the other hand, the amounts of riboflavin and pyridoxine in the composition of Yantarin-Sport exceed the Reference Daily Intake (RDI) more than tenfold. A systematic intake of excessive quantities of these vitamins is fraught with the development of allergic or other undesired reactions. Excess Vitamin B2 may cause damage to renal tubules and fluid retention in the body. Excess Vitamin B6 may lead to the development of anemia, impaired coordination and numbness of the limbs.

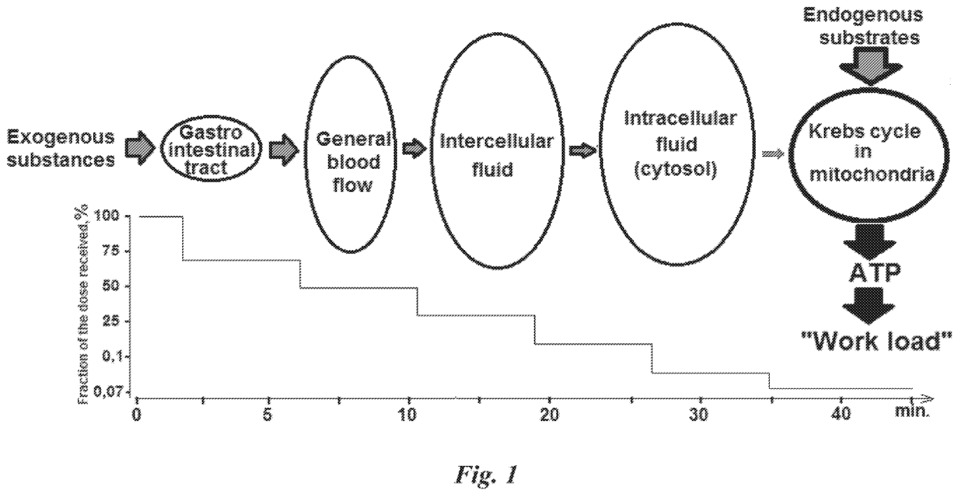

In summary, the aforementioned prototype and its counterparts have drawbacks, such as unpredictability, an insufficient expression of the end effect, and a local irritant effect. The said agents' insufficient activity and local irritant effect are based on delayed absorption and a significant loss of ingredients in the gastrointestinal tract. Considering the decrease of the administered components' concentrations as they pass through the gastrointestinal tract, liver, pulmonary circulation, and finally into the left ventricle and the bloodstream, less than tenths of the taken metabolites reach the brain and effector peripheral tissue (FIG. 1).

The formulations and dosage forms of the present disclosure reduce and eliminate these shortcomings by: first, using ingredients capable of reproducing the triggering of natural adaptive responses that develop during HIPC; second, selecting a ratio of anions and cations that enables a sufficiently rapid release of the administered metabolites into the bloodstream. As a result, the loss of ingredients and irritation of the gastrointestinal mucosa are minimized and a short-term gain of the administered components over their usual concentration in the bloodstream is achieved. The latter creates a metabolic shift that facilitates the triggering of a number of adaptive processes.

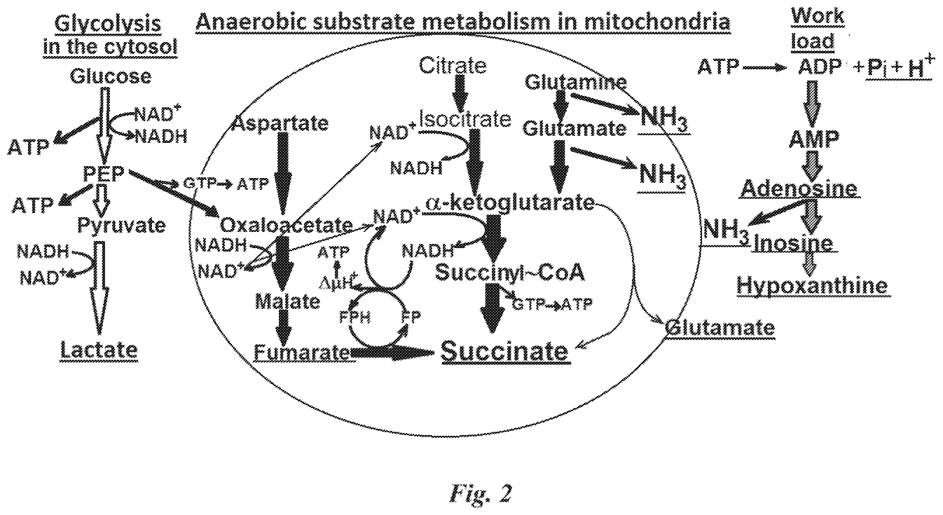

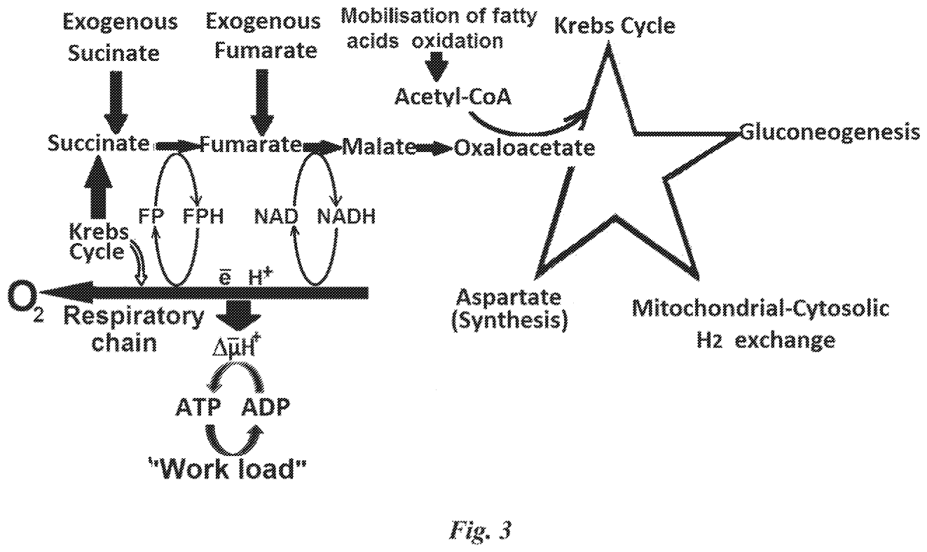

Short hypoxic and anaerobic stresses are accompanied by the mitochondrial and cellular release of metabolites that perform not just the substrate function, but the signaling and regulatory functions as well. These metabolites are released into the intercellular space and the bloodstream and trigger adaptive processes. Under stress, the low-energy shift initiates the decay of a portion of adenylic nucleotides (ATP, ADP, AMP) and the engagement of amino acids into catabolism. Both of these phenomena lead to the production and increased release of ammonia, ammonium ion, inorganic phosphate and hydrogen ions from cells [1, 18, 19] (FIG. 2). Hypoxic stimulation of glycolysis is accompanied by the release of lactate together with hydrogen ions. The semi-anaerobic and anaerobic transformation of the tricarboxylic acid (TCA) cycle provides for the mitochondria's release of succinate, fumarate and glutamate [20]. Stress mobilization of lipids during hypoxia is accompanied by the accumulation of under-oxidized fatty acids and their CoA derivatives. Accordingly, formulations and dosages forms of the present disclosure comprise metabolites that accelerate the complete oxidation of the under-oxidized products of fatty acid oxidation, above all the source of oxaloacetate [FIG. 3]. The latter promotes the involvement of acetyl-CoA, the end product of fatty acids' beta-oxidation, in the TCA cycle [21]. The source of oxaloacetate in the TCA cycle is succinate and fumarate, as well as pyruvate in the form of ammonium, sodium or magnesium salts, and free succinic acid. Meanwhile, carnitine fumarate serves as the source of both fumarate and L-carnitine.

Sodium fumarate is an easily soluble compound and is therefore widely used in the composition of anti-hypoxic resuscitation infusion products such as Mafusol, Polyoxifumarin and Konfumin [22]. The infusion of these products improves hemodynamics, cardiac output and indicators of catabolism, reduces the extent of oxidative stress, and supports mitochondrial performance [23]. A feature of most fumarate-containing infusion agents designated for the treatment of shock or for resuscitation is especially high doses of sodium fumarate in the range of 15 g per 1 liter to 15 g per 0.1 liter.

It is recommended that dietary supplements contain significantly lower doses of fumarate in the form of sodium (E365), potassium (E366), calcium (E367) or ammonium (E368) salts, as well as in the form of iron fumarate [24]. In addition to medicinal products, dozens of products containing fumarate and other carboxylic acids, amino acids and antioxidants have been developed, including dietary supplements, food products, feed premixes, flavor enhancers, alcoholic beverages, softeners and disinfectants, sweeteners, and even agents for protecting plants and increasing crop yields.

Catabolic biochemical transformations of fumarate depend on the tissue redox state. Under aerobic conditions, fumarate quickly passes through the Krebs cycle into oxaloacetate (FIG. 3). Negligibly small concentrations of oxaloacetate, about several micromoles, activate gluconeogenesis and stimulate the oxidation of acetyl-CoA generated under the stress mobilization of lipids, thereby reducing the damaging effect of the excess of under-oxidized fatty acids and their acyl-CoA on membranes and multi-enzyme complexes [25]. The same mechanism promotes oxidation and inhibits the excessive production of ketone bodies, cholesterol and lipids, and prevents the production of the non-specific mitochondrial lipid pore responsible for apoptosis [26].

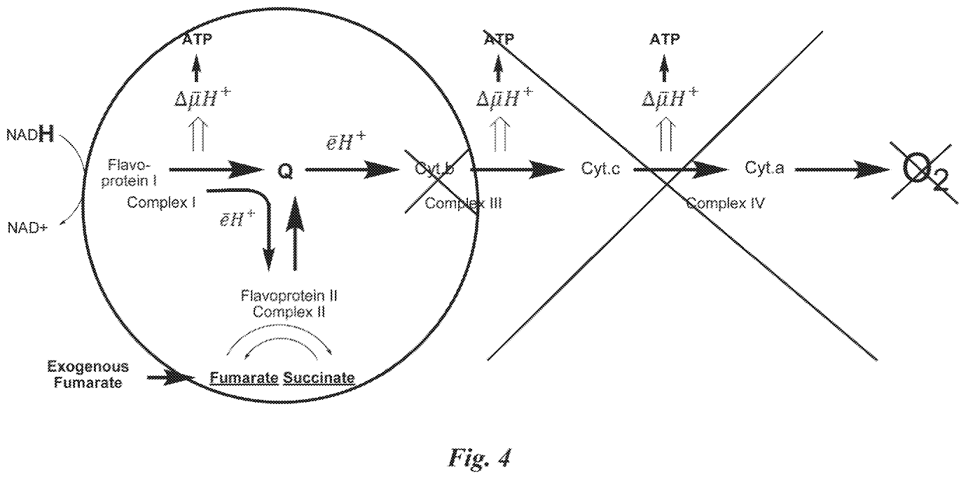

In hypoxia and ischemia, fumarate is a source of succinic acid (FIG. 4). Succinate formed from fumarate in a fumarate reductase reaction with oxygen deficiency performs a signaling function. Succinate, in turn, interacts as a ligand with the specific succinate receptor SUCNR1 [27] and functions as a stabilizer of the transduction hypoxia-inducible factor HIF [28].

Thus, the anti-hypoxic effect of fumarate- and succinate-containing formulations substrate concentrations is associated with fumarate's participation in anaerobic succinate production reactions, in the Krebs cycle circuit, and with the known advantages of succinate oxidation in hypoxia compared to any NAD-dependent substrates [29, 30, 31].

Under anaerobic conditions, fumarate serves as the final hydrogen acceptor at the coenzyme Q level, reverting to succinate. This process makes it possible to sustain the flow of reducing equivalents and enables the generation of the transmembrane electrochemical potential of hydrogen ions and ATP resynthesis at the level of the first respiratory chain complex (FIG. 4), even under conditions of total anaerobiosis or inhibition of the respiratory chain's terminal segment [20, 31].

As soon as an anaerobic state turns hypoxic, succinate is oxidized to fumarate via Complex II, and Complexes III and IV are activated with the generation of the transmembrane electrochemical potential of hydrogen ions and ATP resynthesis.

The preference of succinate over NAD-dependent intermediates in oxidation is due to the fact that, in hypoxia, the initial NAD-dependent segment of the respiratory chain is almost completely reduced while the respiratory chain's terminal segment and flavoproteins (FP) (FIG. 3) are still oxidized to a large extent. In other words, the path for the reducing equivalents is blocked from the NAD-dependent segment, while succinate, as a flavin-dependent substrate, can still continue to oxidize. Therefore, the oxidative phosphorylation of the respiratory chain at levels III and VI is sustained, despite the fact that NADH cannot be fully oxidized.

A similar situation the preferential oxidation of succinate in hypoxia against the background of the inhibited oxidation of NAD-dependent substrates is well known [29, 30, 31]. In anaerobiosis, a radioactive label from NAD-dependent substrates is accumulated in succinate [32, 33]. If oxygen is present, the oxidation of succinate occurs first. At the same time, in hypoxia the concentration of succinate in tissues can decrease almost twofold [34] relative to normoxic conditions due to succinate's preferential oxidation under hypoxia.

Both in vitro and in vivo, when hypoxic and anoxic states alternate in time or space, a succinate-fumarate cycle is formed. In anaerobic “areas,” the NADH-fumarate reductase reaction enables the accumulation of succinate. In hypoxic “areas,” the residual functioning of Complexes III and IV promotes the oxidation of succinate to fumarate. The succinate-fumarate cycle has been recorded at the level of the entire body between the lungs and peripheral tissues [35]. The presence of the pO₂ gradients between the cells in the tissue and between the mitochondria in the cell is based on the difference in distances between the cells and capillaries and between the mitochondria and external cell membrane. This inevitably leads to the formation of the succinate-fumarate cycle. This “cooperation” of the mitochondria, cells and organs at the level of metabolic transformations is a natural mechanism to sustain the viability of the organelles, cells and tissues under the conditions of a natural, pronounced heterogeneity of intracellular and tissue pO₂. Therefore, the additional supply of exogenous succinate and fumarate plays an energy-supporting role, sustaining natural biochemical mechanisms.

Maintaining the NADH-fumarate reductase reaction by supplying exogenous fumarate is especially important during the end period of the oxygen-deficient states before rest/recovery following intense stresses. The oxidation of exogenous and endogenous succinate and the metabolic transformation of fumarate in hypoxia before the onset of normoxia are difficult to reassess during the recovery period. Post-stress, there is a need to eliminate the energy deficiency, which to a large extent can be achieved by the robust oxidation of succinate formed from endogenous sources or administered externally. These phenomena have been repeatedly demonstrated experimentally and clinically during the resuscitative infusion of succinate- and fumarate-containing agents.

It seems that low doses of fumarate and succinate should not be able to sustain energetics. Yet, if their supply to cells is at the level of 10⁻⁴ mol, then it is sufficient to sustain the micromolar pool of oxaloacetate, the level of which does not exceed 10⁻⁴+10⁻⁵ mol even in normoxia. In turn, the development of oxaloacetate has a positive impact on the triggering of the TCA cycle due to the binding of acetyl-CoA in the citrate synthase reaction. As a result, the cleanup of lipid mobilization products is simplified, and fatty acids are oxidized more fully in beta-oxidation. L-carnitine fumarate is introduced into the formulations and dosage forms of the present disclosure for the purpose of fully utilizing this effect. L-carnitine activates the transport of fatty acids into the mitochondria, while fumarate supports the reproduction of oxaloacetate.

As stated earlier, administering dietary supplements through the gastrointestinal tract makes it difficult to reach the substrate concentrations of metabolites in the bloodstream. The FIG. 1 diagram illustrates why the level of metabolites in the bloodstream cannot exceed fractions of a millimole when their high millimolar concentrations are administered through the gastrointestinal tract. This is due to the fact that the interstitial fluid volume exceeds the circulating blood volume by at least tenfold. Metabolites that are diluted even further are supplied to the cells because the concentration of intermediates in the extracellular fluid can barely exceed 5·10⁻⁵ mol.

An “end-to-end” ultra-rapid supply of administered metabolites from the gastrointestinal tract to the bloodstream and tissues is not possible. Delays and losses in the gastrointestinal tract are unavoidable. This is in part due to the partial utilization of the intermediates by mucosal cells and microbiota. Following their absorption from the gastrointestinal tract's lumen, exogenous intermediates proceed along the mesenteric vessels through the portal vein to the liver, where they become rapidly involved in various intracellular metabolic transformations. Next the remaining portion travels through the inferior vena cava to the pulmonary circulation: into the right atrium, ventricle and lungs. The metabolism of succinate, fumarate and pyruvate in these tissues is distinguished by its high rate, as was demonstrated back in 1958 [36]. The administered metabolite portion that had gone through the pulmonary circulation enters the systemic heart, where it is intensely oxidized. From there, it enters the systemic circulation: the aorta, carotid arteries and other vessels that above all feed the brain, kidneys and so on. As a result, the gain in succinate, fumarate and pyruvate concentrations in the systemic circulation from administered agents is not large. The components supplied through the gastrointestinal tract effect little change on the substances' metabolic profiles in the bloodstream, at least not to the extent needed to substantially accelerate catabolic and anabolic transformations. The gradient of the metabolites from the gastrointestinal tract to the mitochondria is at least two orders of magnitude, i.e., 100-fold (see FIG. 1 Diagram). The quantity of exogenous substrates that reaches the mitochondria is much smaller than and not comparable to the massive supply of endogenous substrates that are produced in intracellular metabolic reactions and that enter the TCA cycle even under conditions of rest. As a reminder, under stress, the utilization of endogenous and exogenous substrates increases almost tenfold [1, 19].

Our experimental research with substrates enriched with labeled isotopes (¹³C ¹⁴C) has demonstrated that following the intake of metabolites in doses equivalent to their concentrations in the proposed agent, the amount of exhaled ¹³CO₂ (out of that received with the exogenous substrates) does not exceed fractions of one percent relative to the total pool of exhaled ¹²CO₂, produced from the endogenous non-labeled substrates. Consequently, the observed physiological effects of administering low doses of exogenous substrates are weakly linked to their immediate impact on and participation in catabolism as supplementary fuel. Widely replicated traditional (textbook) notions about the substrate role of metabolic intermediates administered through the gastrointestinal tract have been formed based on in vitro data, specifically during the oxidation of millimolar concentrations of the substrates (>10⁻³ mol) isolated by the mitochondria. These data can hardly be extrapolated to the level of the entire body. This makes it clear that the leading mechanism of the effect of exogenous substrates, in particular fumarate and succinate, in the low doses we have used, may be a signal-regulating action.

Succinate in micromolar concentrations (on the order of 10⁻⁴÷10⁻⁵ mol) is a ligand of the QRP91 X orphan receptor, currently known as SUCNR1 [27, 37]. In addition, an increase of succinate in the tissues acts as a stabilizer of the transcription hypoxia-inducible factor (HIF1α) [28]. Activation of the SUCNR1 receptor, widely present in various types of cells and tissues, may be accompanied by an increased concentration of intracellular calcium. The latter leads to the stimulation of a multitude of metabolic processes and to an increase in the functional activity of the cells. As a reminder, SUCNR1 activation in specific receptor areas of the endothelial cells of the kidneys' juxtaglomerular apparatus's arteries initiates the release of rennin. This process initiates the increase of arterial pressure necessary for the engorgement of the capillary network, i.e., to increase the number of functioning capillaries, improve microcirculation and enhance blood supply to the tissues. On the other hand, the expression and stabilization of HIF1α by an increased level of succinate facilitate the formation of the HIF dimer. In turn, an active HIF dimer initiates the accord transcription of genes responsible for the synthesis of the glucose transporter, glycolysis enzymes, erythropoietin, and the factors enabling the sprouting of new blood vessels and so on.

It is worth noting that, given succinate's normal level of 2±80 μmol, its concentration must be increased to 150±180 μmol in order to activate SUCNR1 or to stabilize HIF. An elevated level of succinate in the bloodstream is indicative of pathologies such as pronounced type 2 diabetes, chronic tissue ischemia, resistant hypertension or the presence of an inherited succinate dehydrogenase (SDH) deficiency. Inherited SDH deficiencies are characteristic of several neogeneses (pheochromocytoma, paraganglioma, etc.). The effects of a continuously pathologically elevated level of endogenous succinate are not comparable to the effects of brief, several-second-long “pulses” of increased succinate concentration, which occur during exercise, intermittent episodes of hypoxia/ischemia, or diving. The conflation of these phenomena under the same “umbrella” of stresses achieves nothing but confusion, fear of real and perceived danger, and misunderstanding.

A spike in the concentration of succinate outside of mitochondria in excess of tens of micromoles is a signal to trigger a set of processes necessary for the mobilization of functions, and above all, it contributes to an enhanced tolerance of the tissues and the entire body to high stresses and oxygen deficiency. This mobilization is phylogenetically anchored by ancient adaptation mechanisms at the receptor and genetic level and can be initiated by the process of physiological and/or metabolic preconditioning.

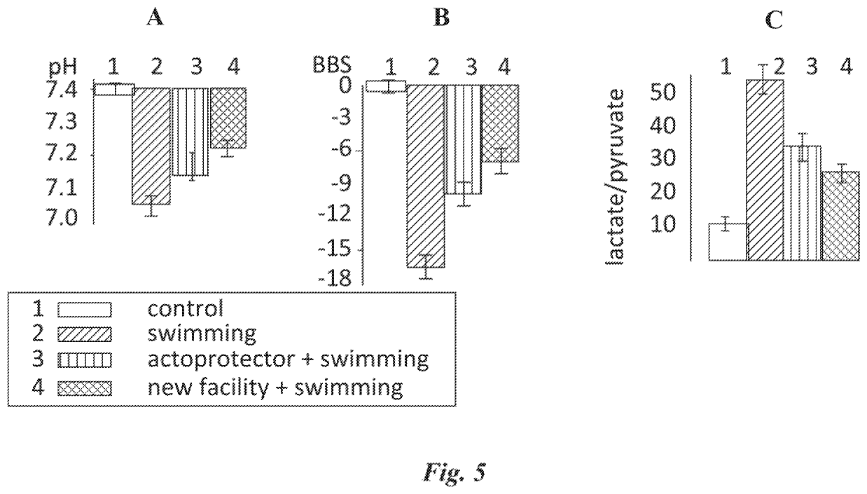

In summary, the appearance of succinate outside of mitochondria at the level of 10⁻⁴ mol and above in conditions of oxygen deprivation is due to a fundamental change of the metabolic transformations in the TCA cycle. As a result, succinic acid becomes the final product of the TCA cycle under ischemia, as described earlier (FIG. 2) [20, 31, 32, 33]. Anaerobic glycolysis, together with the anaerobic production of succinate, cannot fully compensate for ATP consumption under active stress. Therefore, a portion of ATP degrades to ADP and AMP and beyond (FIG. 5). All ATP degradation products, including inorganic phosphate, ammonia and ammonium ions, are released from the cell. This is conducive to the neutralization of unoxidized lactic and succinic acids, which can be released from cells either in the protonated form or in the form of ammonium salts. An increase in the concentration of succinate in the interstitial fluid and blood to a level of 2.10⁻⁴ mol is sufficient for both the activation of the SUCNR1 receptor and the stabilization of the HIF dimer, with all the ensuing consequences.

For this exact reason, the fumarate and succinate contained in the formulations and dosage forms provided herein combine with ammonium and phosphate ions. In this way, the primary elements of the metabolic momentum of anoxic preconditioning are modeled. As a result, the tissues and body as a whole are prepared for subsequent intense stresses.

An important condition for achieving a signal spike in the succinate anion concentration in the bloodstream is the rapid supply of exogenous succinate, fumarate and pyruvate into the bloodstream, while minimizing losses in the gastrointestinal tract. To increase the bioavailability of succinate, fumarate and pyruvate anions, the mechanisms of symport with ammonium, sodium and hydrogen cations are preferably sustained.

To minimize the loss of the formulation ingredients in the gastrointestinal tract, two approaches are used: 1) acceleration of ingredient absorption (transport) through the membrane barriers of the gastrointestinal tract cells and 2) inhibition of the gastrointestinal microbiota's permeases. (Permeases use microorganisms to absorb mono- and dicarboxylic acids.)

The acceleration of the transport of mono- and dicarboxylic acids is achieved by sustaining the primary elements of the organic acid anion transport mechanism through the cell membrane. If sufficient energy-dependent Na⁺/K⁺ transmembrane gradient is present, mono- and dicarboxylic transporters can work in symport with Na⁺ and K⁺. During de-energizing, mono- and dicarboxylic anions are transported in symport with an ammonium ion or proton by means of simple diffusion or uniport through the lipid bilayer [38, 39], and finally by means of antiport in exchange for a lactate or malate anion, for example. In other words, the formulation ensures the functioning of membrane transport systems almost independently of the energy state of the cells. Inhibiting the bacterial permeases ensures that an ammonium ion is present.

An important factor contributing to the metabolic composition's effect is the presence of a “remote” preconditioning mechanism that is unconstrained by the local effect of metabolites due to their entrance into the bloodstream.

Thus, the succinate, fumarate, pyruvate and L-carnitine fumarate in the formulations of the present disclosure contribute to the maintenance of a deficient micromolar pool of oxaloacetate and to full catabolic functioning. With a deteriorating oxygen supply, these components of the formulation are sources of succinate the SUCNR1 succinate receptor ligand and hypoxia-inducible factor (HIF) stabilizer. In this way, key elements of aerobic and anaerobic catabolism are supported at various levels, from glycolysis and mitochondria to the receptor activation of cells and the triggering of adaptive gene expression. A combination of these factors promotes the mobilization and activation of recovery processes.

Presently, the requirements and instructions for administering dietary supplements and medications are established based on statistically significant data on safety and effectiveness. However, a statistical probability cannot be an appropriate criterion for generating individual predictions. Statistical significance is far from equivalent to clinical significance [40]. Moreover, the extrapolation of data obtained from animal experiments is not always suitable for humans. Here is a telling example of increasing the concentration of succinate in the bloodstream. Under certain conditions, an increase in succinate concentration in a number of different pathologies causes an increase in the arterial pressure in animals. However, under the same conditions, it does not have the same hypertensive activity in humans [41]. This may be related to the substantially lower stationary level of succinate in humans than in rodents. It is possible that the process of anaerobic production of succinate in TCA cycle reactions in human tissues is lower than in rodents. Unlike humans, rodents, who frequently lead a “semi-anaerobic” lifestyle, are more tolerant to hypoxia/ischemia. Our research has confirmed the data of Sadagopan et al. [41] we have not found our succinate-containing composition to have any manifested hypertensive effect.

One embodiment of a formulation of the present disclosure is the usage of various conformers of succinic acid in its composition. The natural conformer and salts of succinic acid have a biological activity several times greater than that of chemically synthesized succinate. Moreover, the natural conformer has an anti-carcinogenic and positive cardiotropic effect [42]. We have demonstrated that the natural conformer of succinic acid contributes to the prevention of severe intoxication from four- to seven-day alcohol poisoning or acute barbiturate poisoning.

A feature of compositions containing ammonium succinate is their maintenance of the endocrine apparatus's function (in the hypothalamic-pituitary-ovarian axis) [43], which provides relief for most pathological symptoms during menopause when the Amberen dietary supplement is administered [44, 45]. We have demonstrated experimentally that compositions containing ammonium succinate can be used in conjunction with other agents to achieve an apparent anti-stress effect. For now, it is rather difficult to reconcile these data with the perception of SUCNR1 as a “stress receptor.”

As demonstrated by the present disclosure, the inclusion of small doses of antioxidants in the formulations provided herein contributes to supporting antioxidant systems, not replacing them. The strain on antioxidant systems usually increases with stresses and is especially pronounced during extremely intensive physical work, due to the excessive generation of active forms of oxygen and nitrogen. This process is necessary for the adaptation to physical stresses. Under such conditions, using large replacement doses of antioxidants may be accompanied by a development of injuries rather than by an adaptive effect [46]. Maintaining antioxidant systems with small doses is not only effective during stresses it is necessary during an age-related or pathological weakening of the antioxidant systems.

Embodiments for administering different variations of the formulations provided herein as follows.

In some embodiments, the a formulation according to one of the embodiments provided herein is tested to determine the type of bodily response to the formulation. The user takes a test dose of the formulation (calculated as 3-4 mg per 1 kg of weight) and records the subjective sensations experienced over two hours under regular conditions, without any additional stresses. If the user experiences a burst of energy or a local rush of blood, or if a subjectively noticeable effect is absent, the formulation can be recommended for use before stress to enhance performance. If drowsiness develops, the formulation is to be used during the post-stress rest period or prior to sleeping to ensure full recovery.

1. To enhance performance, it is recommended to administer the formulation 15-40 minutes before a routine stress in the amount of 5-20 mg per 1 kg of body weight per single dose.

2. To accelerate recovery before a repeated stress, it is recommended to administer the formulation at a dose of 5-10 mg per 1 kg of body weight after the first stress if the rest period before the repeated stress is at least 2-3 hours. For a more complete recovery before a repeated stress the following day, the formulation is to be administered after dinner. A longer rest period allows for a short course of the formulation for 3-5 days.

3. A complete course of the formulation promotes a long-lasting performance enhancement effect and recovery acceleration after the course is concluded. This includes daily administration of the proposed formulation for 2-4 weeks. Combining the course administration with a training program increases the effect of the treatment. Once the course of the formulation is concluded, the said effect lasts for at least 2-4 weeks.

The effectiveness of various embodied formulations on animals running on a treadmill and swimming with weights, as well as on athletes of different disciplines and skill levels under mental, psycho-emotional and intense physical stresses of aerobic, anaerobic and mixed types are provided herein.

A reduction in acidosis has been documented for exercises of normal intensity, as well as for moderate aerobic and submaximal, combined aerobic-anaerobic exercises. Administering the formulation during combined aerobic and anaerobic, maximal endurance step exercises promotes the tolerance of deeper acidosis and significantly improves performance despite deepening acidosis. There develops an adaptive tolerance to an acid pH-shift of a magnitude impossible to imagine under different conditions [47, 48]. Moreover, the more intense the exercise is, the more pronounced the effect of the formulation becomes.

Older people may also have two types of responses. The most frequently observed is increased mental and physical activity. Drowsiness occurs less frequently. With both types of responses, a more complete recovery following stresses and sleep has been noted, as well as an easier time performing subsequent work.

Without wishing to be bound by theory, the formulation's effectiveness, specifically with respect to performance enhancement and the acceleration of recovery from fatigue following intense psycho-emotional, mental and physical stresses, depends on a number of factors. Above all, it has to do with the increased bioaccessibility of the formulation's active components. This, in turn, allows the key elements of the natural metabolic state of HIPC to be reproduced.

Different variations of formulations are composed of natural catabolic intermediates: succinic, fumaric and pyruvic acids, their sodium, magnesium, ammonium, calcium and potassium salts, as well as L-carnitine fumarate, antioxidants, ammonium phosphate and sodium bicarbonate.

The formulations provided herein are active as a single dose or a short-term course. A more stable effect is achieved with daily administration for a period of 2-4 weeks. Important characteristics of the formulations and their effects are the usage of small concentrations of natural metabolites, the lack of habit formation, the preservation and increase of the body's energy resources and reserves at a high safety level.

The present disclosure provides formulations, dosage forms and uses thereof for enhancing physical performance and recovery. In particular, the instant disclosure relates to unique formulations comprising ammonium succinate and other active ingredients useful to support physical activity and recovery from physical activity in a subject. In certain embodiments, formulations and dosage forms provided herein may be used for: improving or enhancing physical performance; improving or enhancing recovery from physical performance; improving or enhancing tolerance to stress; improving or enhancing tolerance to change in time zones (jet lag); improving or enhancing quality of sleep; decreasing fatigue; improving or enhancing alertness or focus; improving or enhancing sense of well-being; improving or enhancing energy level or motivation; improving or enhancing sense of relaxation or well-being; improving or enhancing tolerance to stress; decreasing anxiety or irritability; or any combination thereof.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate fractions thereof (such as one tenth and one hundredth of an integer), unless otherwise indicated. As used herein, the term “about” means±20% of the indicated range, value, or structure, unless otherwise indicated. The term “consisting essentially of” limits the scope of a claim to the specified materials or steps, or to those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

As used herein, “subject” refers to a mammal. Mammals include humans, primates, cows, horses, goats, sheep, dogs, cats, mice, rats, rabbits, guinea pigs, pigs, and transgenic species thereof.

As used herein, “active ingredient” or “active agent” refers to a compound that induces a desired pharmacological, or physiological effect. An active ingredient also encompass pharmaceutically acceptable, pharmacologically active derivatives of the active ingredients disclosed herein, including, but not limited to, salts, esters, amides, prodrugs, active metabolites, analogs, and the like. In certain embodiments, compositions of the present disclosure may contain multiple active ingredients that may have the same or different pharmacological or physiological effects.

“Effective amount” or “therapeutically effective amount” refers to that amount of a formulation of this disclosure which, when administered to a mammal, is sufficient to effect a desired biological effect or treatment. The amount of a formulation of this disclosure that constitutes an “effective amount” will vary depending on the formulation, the manner of administration, the duration of treatment, or the age, body weight, general health, diet or activity level of the subject being treated but can be determined routinely by one of ordinary skill in the art having regard to his own knowledge and to this disclosure.

The terms “physical performance”, “exercise,” “exertion,” and “work” refer to physical activity by a subject. The physical activity may be aerobic or anaerobic. The physical activity may include, for example, one or more of walking, running, sprinting, swimming, hiking, climbing, skating, weight training, bicycling on either a stationary or non-stationary bicycle, boxing in a match or using sandbags or other training equipment, rowing on either a rowing simulator or in a boat, using cardio exercise machines, dancing, wrestling, martial arts, weight lifting, or participating in any type of sport. The physical performance may comprise multiple activities, such as participation in a sport or other activity that incorporates multiple forms of physical activity, for example running and jumping. In certain embodiments, physical activity is a moderate aerobic activity, moderate anaerobic activity, intense aerobic activity, intense anaerobic activity, or a combination thereof. The physical activity may be a competitive event, a non-competitive event, or a training event.

“Cycloergometric exercise” refers to exercise on a stationary or non-stationary bicycle. Such exercise may be either aerobic, anaerobic, or mixed aerobic-anaerobic depending on the speed of pedaling, resistance to pedaling, and duration of the exercise.

### Pharmaceutical Formulations and Dosage Forms

In one aspect, the present disclosure provides a first pharmaceutical formulation comprising the active ingredients: ammonium succinate, sodium succinate, succinic acid, potassium fumarate or sodium fumarate, ammonium phosphate, and L-carnitine fumarate or a mixture of L-carnitine and sodium fumarate at a 1:1 ratio (L-carnitine:sodium fumarate) (also referred to as the “Active” Formulation). In one embodiment, the active ingredients of the first formulation comprise about 30%-45% ammonium succinate, about 5%-20% sodium succinate, about 5%-15% succinic acid, about 10%-20% potassium fumarate or sodium fumarate, about 5%-20% ammonium phosphate, and about 10%-20% L-carnitine fumarate or a mixture of L-carnitine and sodium fumarate at a 1:1 ratio. In one embodiment, the ratio of ammonium succinate, sodium succinate, succinic acid, potassium fumarate or sodium fumarate, ammonium phosphate, and L-carnitine fumarate or L-carnitine: sodium fumarate are about 4-6:0.3-2.5:0.3-2.5:0.6-2.5:0.5-2.5:0.6-2.5, respectively. In one embodiment, the ratio of ammonium succinate, sodium succinate, succinic acid, potassium fumarate or sodium fumarate, ammonium phosphate, and L-carnitine fumarate or L-carnitine:sodium fumarate is about 5:1:1:2:1:2, respectively. In one embodiment, the active ingredients comprises about 41% ammonium succinate, about 8% sodium succinate, about 8% succinic acid, about 17% potassium fumarate, about 8% ammonium phosphate, and about 17% L-carnitine fumarate. The percentages provided with reference to total amount active ingredients does not include pharmaceutical excipients or carriers that may be present in the formulation. In certain embodiments, the sodium succinate is in the form of sodium succinate dibase hexahydrate.

In certain embodiments, the first formulation (“Active Formulation”) comprises about 24%-36% by weight ammonium succinate, about 4%-16% by weight sodium succinate, about 4%-12% by weight succinic acid, about 8%-16% by weight potassium fumarate or sodium fumarate, about 2%-8% by weight ammonium phosphate, and about 8%-16% by weight L-carnitine fumarate or a mixture of L-carnitine and sodium fumarate at a 1:1 ratio. In one embodiment, the active ingredients of the first formulation are at an about 4-6:0.3-2.5:0.3-2.5:0.6-2.5:0.5-2.5:0.6-2.5 ratio, respectively. In one embodiment, the active ingredients of the first formulation are at an about 5:1:1:2:1:2 ratio, respectively. In one embodiment, the first formulation comprises about 34% by weight ammonium succinate, about 7% by weight sodium succinate, about 7% by weight succinic acid, about 14% by weight potassium fumarate, about 7% by weight ammonium phosphate, and about 14% by weight L-carnitine fumarate. In one embodiment, sodium succinate is sodium succinate dibase hexahydrate. In one embodiment, the first formulation further comprises a pharmaceutical excipient. In one embodiment, the pharmaceutic excipient comprises a filler, a lubricant, an anticaking agent, or any combination thereof. In one embodiment, the first formulation comprises filler in an amount of about 10%-20% by weight. In one embodiment, the first formulation comprises lubricant in an amount of about 1%-5% by weight. In one embodiment, the first formulation comprises anticaking agent in an amount of about 1%-5% by weight.

In another aspect, the present disclosure provides a second pharmaceutical formulation comprising the active ingredients: ammonium succinate, sodium succinate, and ascorbic acid (also referred to as the “Pro-Sport” Formulation). In one embodiment, the active ingredients in the second formulation comprise about 61%-85% ammonium succinate, about 7%-15% sodium succinate, and about 7%-15% ascorbic acid. In one embodiment, the ratio of ammonium succinate, sodium succinate, and ascorbic acid is about 8-11:1-2:1-2, respectively. In one embodiment, the ratio of ammonium succinate, sodium succinate, and ascorbic acid is about 10:2:1, respectively. In one embodiment, the active ingredients comprises about 77% ammonium succinate, about 15% sodium succinate, and about 7% ascorbic acid. The percentages provided with reference to total amount active ingredients does not include pharmaceutical excipients or carriers that may be present in the formulation. In certain embodiments, the sodium succinate is in the form of sodium succinate dibase hexahydrate.

In certain embodiments, the second formulation (“Pro-Sport Formulation”) comprises about 54%-76% by weight ammonium succinate, about 6%-14% by weight sodium succinate, and about 6%-14% by weight ascorbic acid. In one embodiment, the active ingredients of the second formulation are at an about 8-11:1-2:1-2 ratio, respectively. In one embodiment, the active ingredients of the first formulation are at an about 10:2:1 ratio, respectively. In one embodiment, the second formulation comprises about 70% by weight ammonium succinate, about 14% by weight sodium succinate, and about 11% by weight ascorbic acid. In one embodiment, sodium succinate is sodium succinate dibase hexahydrate. In one embodiment, the second formulation further comprises a pharmaceutical excipient. In one embodiment, the pharmaceutic excipient comprises a lubricant, an anticaking agent, or both. In one embodiment, the second formulation comprises lubricant in an amount of about 1%-5% by weight. In one embodiment, the second formulation comprises anticaking agent in an amount of about 1%-6% by weight.

In yet another aspect, the present disclosure provides a third pharmaceutical formulation comprising the active ingredients: ammonium succinate, sodium succinate, succinic acid, potassium fumarate or sodium fumarate, ammonium phosphate, L-carnitine fumarate or a mixture of L-carnitine and sodium fumarate at a 1:1 ratio, an antioxidant, dihydroquercetin, α-tocopherol, and ascorbic acid (also referred to herein as “Sentinel Formulation”). In one embodiment, the active ingredients in the third formulation comprise about 41%-54% ammonium succinate, about 8%-16% sodium succinate, about 5%-10% succinic acid, about 5%-8% potassium fumarate or sodium fumarate, about 2%-5% ammonium phosphate, about 9%-16% L-carnitine fumarate or a mixture of L-carnitine and sodium fumarate at a 1:1 ratio, about 1%-3% antioxidant, about 5%-11% dihydroquercetin, about 5%-11% α-tocopherol, and about 4%-16% ascorbic acid. In one embodiment, the ratio of the active ingredients of the third formulation is about 8.6-11.33:1.6-3.33:0.99-1.99:0.97-1.66:0.46-0.92:1.9-3.32:0.3-0.6:0.99-1.99:0.99-1.99:1-3.33, respectively. In one embodiment, the ratio of the active ingredients of the third formulation is about 10:2:1.13:1.33:0.53:2.33:0.4:1.13:1.13:1, respectively. In one embodiment, the active ingredients comprise about 48% ammonium succinate, about 10% sodium succinate, about 5% succinic acid, about 6% potassium fumarate, about 3% ammonium phosphate, about 11% L-carnitine fumarate, about 2% antioxidant, about 5% dihydroquercetin, about 5% α-tocopherol, and about 5% ascorbic acid. The percentages provided with reference to total amount active ingredients does not include pharmaceutical excipients or carriers that may be present in the formulation. In certain embodiments, the antioxidant may be curcumin or lycopene. In certain embodiments, the sodium succinate is in the form of sodium succinate dibase hexahydrate.

In certain embodiments, the third formulation (“Sentinel Formulation”) comprises about 36%-48% by weight ammonium succinate, about 7%-14% by weight sodium succinate, about 4%-9% by weight succinic acid, about 4%-7% by weight potassium fumarate or sodium fumarate, about 2%-4% by weight ammonium phosphate, about 8%-14% by weight L-carnitine fumarate or a mixture of L-carnitine and sodium fumarate at a 1:1 ratio, about 1%-3% by weight antioxidant, about 4%-10% by weight dihydroquercetin, about 4%-10% by weight α-tocopherol; and about 4%-14% by weight ascorbic acid. In one embodiment, the active ingredients of the third formulation are at an about 8.6-11.33:1.6-3.33:0.99-1.99:0.97-1.66:0.46-0.92:1.9-3.32:0.3-0.6:0.99-1.99:0.99-1.99:1-3.33 ratio, respectively. In one embodiment, the active ingredients of the third formulation are at an about 10:2:1.13:1.33:0.53:2.33:0.4:1.13:1.13:1 ratio, respectively. In one embodiment, the third formulation comprises about 43% by weight ammonium succinate, about 9% by weight sodium succinate, about 5% by weight succinic acid, about 6% by weight potassium fumarate, about 2% by weight ammonium phosphate, about 10% by weight L-carnitine fumarate, about 2% lycopene or curcumin, about 5% by weight dihydroquercetin, about 7% α-tocopherol, and about 8% ascorbic acid. In certain embodiments, the sodium succinate is in the form of sodium succinate dibase hexahydrate. In one embodiment, the third formulation further comprises a pharmaceutical excipient. In one embodiment, the pharmaceutic excipient comprises a lubricant, an anticaking agent, or both. In one embodiment, the first formulation comprises lubricant in an amount of about 0.1%-5% by weight. In one embodiment, the first formulation comprises anticaking agent in an amount of about 2%-4% by weight.

Ammonium succinate and sodium succinate are the ammonium and sodium salts, respectively, of succinic acid. As used herein, sodium succinate may refer to sodium succinate dibase hexahydrate, anhydrous sodium succinate, or both. Succinic acid may take the form of one of several different conformers. The natural conformer of succinic acid has the highest biological activity, but is difficult to isolate. The term succinic acid may refer to the natural conformer, a conformer other than the natural conformer, or a mixture of conformers. Succinic acid and salts thereof are important elements of several metabolic pathways, including both anaerobic metabolism (FIG. 2) and aerobic metabolism (FIG. 3). Furthermore, sodium succinate may also serve to reduce irritation of the gastro-intestinal tract when combined with ammonium succinate.

Fumarate is an element of both anaerobic and aerobic metabolism. The formulations described herein may comprise fumarate in the form of potassium fumarate, sodium fumarate, L-carnitine fumarate, or a combination thereof. L-carnitine activates the transport of fatty acids into the mitochondria. In certain embodiments, sodium fumarate and/or sodium bicarbonate may be used in place of part or all of the potassium fumarate in order to reduce irritation of the gastro-intestinal tract. However, in subjects with tachycardia, it may be preferable to use potassium fumarate over sodium fumarate.

Ammonium phosphate may take the form of ammonium phosphate monobasic, ammonium phosphate dibasic, or a mixture of thereof.

In certain embodiments, formulations of the present disclosure comprise an antioxidant. In one embodiment, the antioxidant is lycopene. Lycopene can take several different isomeric forms, including all trans, 5-cis, 7-cis, 9-cis, 11-cis, 13-cis, and 15-cis. In one embodiment, lycopene is in the form of the 13-cis isomer. In one embodiment, lycopene is a combination of two or more isomers. In another embodiment, the antioxidant is curcumin. Curcumin may refer to curcumin or derivatives thereof, such as demethoxycurcumin and bisdemethoxycurcumin, also referred to as curcumin-BDM or BDMC. In certain embodiments, the antioxidant comprises curcumin, demethoxycurcumin, bisdemethoxycurcumin, or any combination thereof. Other antioxidants, such as glutathione, β-carotene, and ubiquinol, may be also be used.

In certain embodiments, any of the aforementioned formulations may comprise additional active ingredients. In certain embodiments, the additional active ingredient comprises sodium pyruvate, ammonium-sodium succinate, calcium succinate, sodium bicarbonate, or any combination thereof. Sodium pyruvate is another compound involved in metabolism. In certain embodiments, formulations of the present disclosure comprise sodium pyruvate in an amount ranging from about 1% to about 15% by weight or about 5% to about 10% by weight. Compositions comprising sodium pyruvate may be particularly beneficial when the subject exhibits polyneurotic pain or residual symptoms of neuralgia, such as trifacial or sciatic nerve hypersensitivity to changes in temperature. Ammonium-sodium succinate is a salt of succinic acid having one ammonium ion and one sodium ion. In certain embodiments, formulations of the present disclosure comprise ammonium-sodium succinate in an amount of about 1% to 10% by weight. Calcium succinate is a salt of succinic acid that may be used particularly in cases where extra calcium supplementation is desirable, for example if the subject has suffered a bone fracture. In certain embodiments, formulations of the present disclosure comprise calcium succinate an amount ranging from about 1% to about 10% by weight. Sodium bicarbonate may reduce irritation of the gastro-intestinal tract. In certain embodiments, formulations of the present disclosure comprise sodium bicarbonate in an amount ranging from about 1% to about 7% by weight.

The formulations provided herein may processed into a variety of dosage forms for oral, topical, or parenteral administration to a subject. Typical routes of administration include, without limitation, oral, parenteral, sublingual, bladder wash-out, vaginal, rectal, enteric, suppository, nasal, and inhalation. The term parenteral, as used herein, includes subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, and intraurethral injection or infusion techniques. The dosage forms of the present disclosure are formulated so as to allow the active ingredients contained therein to be bioavailable upon administration of the dosage form to a subject.

In certain embodiments, formulations as described herein may be processed into dosage forms suitable for oral administration to a subject, such as for example, filled capsules, compressed tablets or caplets, or other dosage form suitable for oral administration using conventional techniques. Oral dosage forms are taken orally and release the active ingredients in the gastrointestinal tract. Oral dosages are available in many different solid or liquid forms, including tablet, soft capsule, hard capsule, lozenge, suspension tablet, effervescent tablet, powder, effervescent powder, chewable tablet, suspension, liquid solution, emulsion, gel, cream, syrup, spray, and the like.

Dosage forms typically contain one or more pharmaceutical excipients, which may include inert diluents and edible carriers, in addition to provided active ingredients. Pharmaceutically acceptable excipients for therapeutic use are well known in the pharmaceutical art, and are described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992). Examples of pharmaceutical excipients include binders, fillers, lubricants, glidants, anticaking agents, compression aids, disintegrants, and surfactants.

In certain embodiments, the formulations described herein further comprise a filler, a lubricant, an anticaking agent, or any combination thereof. Exemplary fillers include lactose, calcium carbonate, calcium sulfate, compressible sugars, dextrates, dextrin, dextrose, kaolin, magnesium carbonate, magnesium oxide, maltodextrin, mannitol, microcrystalline cellulose, powdered cellulose, and sucrose. In one embodiment, the filler is microcrystalline cellulose. Exemplary anticaking agents include silicon dioxide, calcium carbonate, calcium phosphate, calcium silicate, calcium stearate, starch, and cellulose. In one embodiment, the anticaking agent is silicon dioxide. Exemplary lubricants include magnesium stearate, stearic acid, calcium stearate, hydrogenated castor oil, hydrogenated vegetable oil, light mineral oil, polyethylene glycol, sodium benzoate, sodium stearyl fumarate, and zinc stearate. In one embodiment, the lubricant is magnesium stearate.

Dosage forms described herein may be manufactured using standard techniques and equipment, for example dry blending, milling, dry granulation, wet granulation, fluid bed granulation, dry powder blending, pelletization, direct pelletization, extrusion, melt-extrusion, spheronization, drug layering, compaction, compression. Suitable methods for the manufacture of the formulations and dosage forms described herein are provided, for example, in Remington, 20^{th} edition, Chapter 45 (Oral Solid Dosage Forms).

In certain embodiments, one or more component of the formulation (i.e., active ingredient) is microencapsulated. Microencapsulation refers to a process by which individual particles of an active ingredient are coated with a continuous hard or soft soluble film to form small particles called microcapsules. Microencapsulation may be used to protect the microencapsulated active ingredient from degradation (e.g., enzymatic, heat, humidity, oxidation), prevent interactions between different active ingredients, or control the release rate of the microencapsulated active ingredient. Materials and methods for microencapsulation may be routinely selected by one of ordinary skill in the art. In one embodiment, two or more active ingredients are microencapsulated together. In one embodiment, two or more active ingredients are microencapsulated separately. In one embodiment, acid salts are separated from other active ingredients, e.g., vitamins, by way of microencapsulation. In one embodiment, ascorbic acid is microencapsulated, α-tocopherol is microencapsulated, ammonium succinate is microencapsulated, or a combination thereof. In certain embodiments, palm oil, soybean oil, cottonseed oil, sunflower oil, or coconut oil is used for microencapsulation. In one embodiment, one or more of the active ingredients is microencapsulated with palm oil.

Exemplary formulations according to the embodiments described herein are provided in Table 1.

TABLE 1
Exemplary Formulations and Ranges

| | Parts | Ratio | | % weight |
|---|---|---|---|---|
| | (Ratio) | Range | % weight | range |
| Exemplary First | | | | |
| Formulation | | | | |
| (“Active”) | | | | |
| Ammonium succinate | 5 | 4-6 | 34.3% | 24%-36% |
| Sodium succinate | 1 | 0.3-2.5 | 6.8% | 4%-16% |
| Succinic acid | 1 | 0.3-2.5 | 6.8% | 4%-12% |
| Potassium fumarate or | 2 | 0.6-2.5 | 13.6% | 8%-16% |
| sodium fumarate | | | | |
| Ammonium phosphate | 1 | 0.5-2.5 | 6.9% | 2%-8% |
| L-carnitine fumarate or | 2 | 0.6-2.5 | 13.6% | 8%-16% |
| L-carnitine:sodium | | | | |
| fumarate at 1:1 | | | | |
| Microcrystalline | | | 14.7% | 10%-20% |
| cellulose | | | | |
| Magnesium stearate | | | 1.5% | 1%-5% |
| Silicon dioxide | | | 2.1% | 1%-5% |
| Exemplary Second | | | | |
| Formulation (“Pro- | | | | |
| Sport”) | | | | |
| Ammonium succinate | 10 | 8-11 | 70.3% | 54%-76% |
| (optionally | | | | |
| microencapsulated) | | | | |
| Sodium succinate | 2 | 1-2 | 14.1% | 6%-14% |
| Ascorbic acid | 1 | 1-2 | 11% | 6%-14% |
| (optionally | | | | |
| microencapsulated) | | | | |
| Magnesium stearate | | | 2% | 1%-5% |
| Silicon dioxide | | | 2.6% | 1%-6% |
| Exemplary Third | | | | |
| Formulation | | | | |
| (“Sentinel”) | | | | |
| Ammonium succinate | 10 | 8.6-11.33 | 43.2% | 36%-48% |
| Sodium succinate | 2 | 1.6-3.33 | 8.6% | 7%-14% |
| Succinic acid | 1.13 | 0.99-1.99 | 4.9% | 4%-9% |
| Potassium fumarate or | 1.33 | 0.97-1.66 | 5.8% | 4%-7% |
| sodium fumarate | | | | |
| Ammonium phosphate | 0.53 | 0.46-0.92 | 2.4% | 2%-4% |
| L-carnitine fumarate or | 2.33 | 1.9-3.32 | 9.9% | 8%-14% |
| L-carnitine:sodium | | | | |
| fumarate at 1:1 | | | | |
| Antioxidant (e.g., | 0.4 | 0.3-0.6 | 1.7% | 1%-3% |
| lycopene or curcumin) | | | | |
| Dihydroquercetin | 1.13 | 0.99-1.99 | 4.8% | 4%-10% |
| α-tocopherol | 1.13 | 0.99-1.99 | 6.9% | 4%-10% |
| (optionally | | | | |
| microencapsulated) | | | | |
| Ascorbic acid | 1 | 1-3.33 | 8.5% | 4%-14% |
| (optionally | | | | |
| microencapsulated) | | | | |
| Magnesium Stearate | | | 0.6% | 0.1%-5% |
| Silicon dioxide | | | 2.8% | 2%-4% |

In certain embodiments, the amount of formulation in the dosage form is about 150 mg 1,700 mg, 250 mg 600 mg, 300 mg 500 mg, 350 mg 400 mg. In certain embodiments, the amount of formulation in the dosage form is about 150 mg, 175 mg, 200 mg, 225 mg, 250 mg, 300 mg, 325 mg, 350 mg, 360 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1025 mg, 1050 mg, 1075 mg, 1100 mg, 1125 mg 1150 mg, 1175 mg 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, or 1700 mg.

For capsule dosage forms, the capsule further comprises a capsule shell. Capsule shells may be comprised of gelatin, hydroxypropyl methylcellulose, starch, pullulan, polyvinyl acetate, or a combination thereof. In one embodiment, the capsule shell comprises hydroxypropyl methylcellulose. When the solid form is a tablet, it may be coated with a film coating using materials and methods known in the art. Film coatings typically contain a sugar, polysaccharide, or polymer, and may also contain a colorant. Such a coating may function to protect the active ingredients from air or moisture, to improve the taste and/or odor of the tablet, or to make the tablet easier to swallow. In one embodiment, the film coating is water resistant. Exemplary water resistant polymers that may be used in film coatings include ethyl cellulose, polyvinyl acetate, polyacrylates, polymethyacrylates. Exemplary water soluble polymers that may be used in film coatings include polyvinyl alcohol, hydroxypropyl methylcellulose, povidone, hydroxypropyl cellulose, hydroxypropyl ethylcellulose, PEG, and hydroxyethylcellulose. In one embodiment, the tablet is coated to 1%-10% weight gain with a film coating.

### Methods of Use

Methods are disclosed herein for treating a subject in need thereof by administering an effective amount of one or more dosage forms as described herein. In various embodiments, conditions or purposes for which the formulations and dosage forms described herein may be administered to a subject include: improving or enhancing physical performance; improving or enhancing recovery from physical performance; improving or enhancing tolerance to stress; improving or enhancing tolerance to change in time zones (jet lag); improving or enhancing quality of sleep; decreasing fatigue; improving or enhancing alertness or focus; improving or enhancing sense of well-being; improving or enhancing energy level or motivation; improving or enhancing sense of relaxation or well-being; improving or enhancing tolerance to stress; decreasing anxiety or irritability; or any combination thereof.

In certain embodiments, a physical performance comprises moderate aerobic exercise, moderate anaerobic exercise, intense aerobic exercise, intense anaerobic exercise, or any combination thereof. In certain embodiments, physical performance is a physical activity (e.g., aerobic or anaerobic exercise) comprising a duration of about 5 minutes, 10 minutes, 15 minutes, 20 minutes, 25 minutes, 30 minutes, 35 minutes, 40 minutes, 45 minutes, 50 minutes, 55 minutes, 1 hour, 1.25 hours, 1.5 hours, 1.75 hours, 2 hours, 2.25 hours, 2.5 hours, 3 hours, 3.5 hours, 3.75 hours, 4 hours, 4.25 hours, 4.5 hours, 4.75 hours, 5 hours, 5.25 hours, 5.5 hours, 5.75 hours, 6 hours, 6.25 hours, 6.5 hours, 6.75 hours, 7 hours, 7.25 hours, 7.5 hours. 7.75 hours, 8 hours, or more. In one embodiment, a physical performance is a single event. In one embodiment, a physical performance is multiple events on the same day. In one embodiment, a physical performance comprises multiple events occurring over continuous days. In one embodiment, a physical performance comprises multiple events occurring of multiple non-continuous days.

In certain embodiments, improving or enhancing physical performance comprises improving or enhancing duration of physical activity. In certain embodiments, an improvement or enhancement of duration of physical activity comprises an increase of about 5%, 10%, 20%, 30%, 40%, 50%, 60%, 70%, 80%, 90%, 100%, 110%, 120%, 130%, 140%, 150%, 160%, 170%, 180%, 190%, 200% or more. In certain embodiments, improving or enhancing physical performance comprises improving or enhancing intensity or workload of physical activity. In certain embodiments, an improvement or enhancement of intensity or workload of physical activity comprises an increase of about 5%, 10%, 20%, 30%, 40%, 50%, 60%, 70%, 80%, 90%, 100%, 110%, 120%, 130%, 140%, 150%, 160%, 170%, 180%, 190%, 200% or more. In certain embodiments, improving or enhancing physical performance comprises increased speed of physical activity. In certain embodiments, an improvement or enhancement of speed of physical activity comprises an increase of about 5%, 10%, 20%, 30%, 40%, 50%, 60%, 70%, 80%, 90%, 100%, 110%, 120%, 130%, 140%, 150%, 160%, 170%, 180%, 190%, 200% or more. In certain embodiments, improving or enhancing physical performance comprises improving or enhancing tolerance of physical activity. Tolerance of physical activity may be measured by decreased heart rate during physical activity, decreased sweating during physical activity, decreased shortness of breath during physical activity, increased resistance to blood acidosis during physical activity.

In certain embodiments, improving or enhancing recovery from physical performance comprises decreasing blood acidosis following physical activity. In certain embodiments, improving or enhancing recovery from physical performance comprises faster slowing of heart rate following physical activity or lowering of resting heart rate. In certain embodiments, improving or enhancing recovery from physical performance comprises improving or enhancing duration, speed, intensity, workload, or tolerance of a subsequent physical activity on the same day, on the following day, within 2 days, within 3 days, within 4 days, within 5 days, within 6 days, or within the same week.

As is well known in the art, therapeutically effective amounts for use in humans can be determined from animal models. For example, a dose for humans can be formulated to achieve a concentration that has been found to be effective in animals. The dosage in humans can be adjusted by monitoring formulations effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the subject and the formulation being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Dosage amounts and intervals can be adjusted individually to provide levels of the administered active ingredients effective for the particular condition or indication being treated.

In certain embodiments, a dosage form according to any of the embodiments described herein is administered to a subject at a dosage of about 3-25 mg/kg of body weight, about 5-20 mg/kg of body weight, about 5-10 mg/kg of body weight, about 8-18 mg/kg of body weight, or about 12-18 mg/kg of body weight. In certain embodiments, a dosage form is administered to a subject at a dosage of about 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, or 25 mg/kg.

In certain embodiments, the dosage form is administered to the subject about 1-3 times a day, once a day, twice a day or three times a day. In certain embodiments, the dosage form is administered to the subject on an intermittent or as needed basis. In certain embodiments, the dosage form is administered to the subject for an initial treatment period of about 2-7 consecutive days, 7-14 consecutive days, 14-28 consecutive days, 2-30 consecutive days, 30-60 consecutive days, 14-60 consecutive days, or 7-60 consecutive days. In certain embodiments, the dosage form is administered to the subject for an initial treatment period of about 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, 35 consecutive days, 40 consecutive days, 45 consecutive days, 50 consecutive days, 55 consecutive days, or 60 consecutive days. In certain embodiments, the initial treatment period is followed by a period of about 2-14 consecutive days in which the dosage form is not administered to the subject before optionally resuming treatment with the dosage form. In certain embodiments, the initial treatment period is followed by a period of about 10-14 consecutive days in which the dosage form is not administered to the subject. In certain embodiments, the initial treatment period is followed by a period of about 2 days, 3 days, 4 days, 5 days, 6 days, 7 days, 8 days, 9 days, 10 days, 11 days, 12 days, 13 days, or 14 days in which the dosage form is not administered to the subject.

In various embodiments, the dosage form is administered to a subject with or immediately following a meal (e.g., within 15 minutes, 30 minutes, 45 minutes or 1 hour of a meal). In certain embodiments, the dosage form is administered to a subject on the same day as a physical performance or stress. In certain embodiments, the dosage form is administered to a subject about 15-90 minutes, 20-60 minutes, or 30-45 minutes before physical performance (exercise) or stress. In certain embodiments, the dosage form is administered to a subject about 15 minutes, 20 minutes, 25 minutes, 30 minutes, 35 minutes, 40 minutes, 45 minutes, 50 minutes, 55 minutes, 60 minutes, 70 minutes, 80 minutes, or 90 minutes before a physical performance or stress. In certain embodiments, the dosage form is administered after physical performance. In certain embodiments, the dosage form is administered in an interval between two or more physical performances.

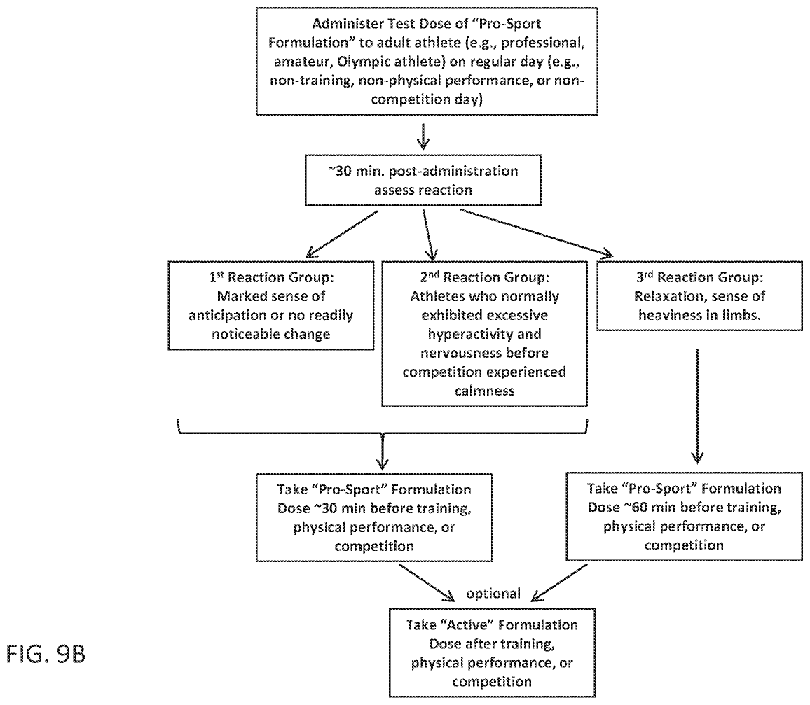

In certain embodiments, a subject is administered a dose of a formulation provided in the present disclosure on a normal day (e.g., not a competition day, training day, physical performance day, etc.) to determine the subject's response to the dosage form. In certain embodiments, the subject is administered one dose of the formulation and initial response to the formulation is assessed after about 20-30 minutes. In certain embodiments, timing of dosage administration is adjusted according to the initial response. Exemplary dosage regimens are provided in FIGS. 9A-9B.

In certain embodiments, the subject is human. In certain embodiments, the subject is an adult. In certain embodiments, the subject is an athlete, including Olympic athletes, professional athletes, and amateur athletes. In certain embodiments, the subject is an active adult (e.g., adults regularly engaged in sports or exercise and active non-athletes). In certain embodiments, the subject is about 18-80 years old, 18-75 years old, 18-70 years old, 18-65 years old, 18-60 years old, 18-55 years old, 18-50 years old, 18-45 years old, 18-40 years old, 18-35 years old, 18-30 years old, 50-80 years old, 55-80 years old, 50-75 years old, 50-70 years old, 60-80 years old, or 65-80 years old.

In certain embodiments, a subject is administered combination therapy comprising two or more different formulations provided herein. In certain embodiments, methods of improving or enhancing physical performance or recovery from physical performance provided herein comprise: (i) administration of an effective amount of a dosage form comprising a first formulation (“Active Formulation”) according to any of the embodiments provided herein to a subject prior to the physical performance and (ii) administration of an effective amount of adosage form of a third formulation (“Sentinel Formulation”) according to any of the embodiments provided herein following the physical performance. In certain embodiments, the dosage form comprising the first formulation is administered to a subject about 15 minutes, 20 minutes, 25 minutes, 30 minutes, 35 minutes, 40 minutes, 45 minutes, 50 minutes, 55 minutes, 60 minutes, 70 minutes, 80 minutes or 90 minutes before the physical performance. In certain embodiments, the dosage form comprising the first formulation is administered to a subject about 15-90 minutes, 20-60 minutes, or 20-30 minutes before the physical performance. In certain embodiments, administration of the dosage form comprising the third formulation is immediately following the physical performance or about 5 minutes, 10 minutes, 15 minutes, 20 minutes, 25 minutes, 30 minutes, 45 minutes, 50 minutes, 55 minutes, 60 minutes, 70 minutes, 80 minutes, 90 minutes, or 2 hours or more after the physical performance. In certain embodiments, administration of the dosage form comprising the third formulation is spaced at least two hours after administration of the dosage form comprising the first formulation. In certain embodiments, the subject is an active adult (e.g., fitness minded adult, adult regularly engaged in sports or exercise, active non-athlete). Exemplary combination therapy dosage regimens are set forth in FIG. 9A.

In certain embodiments, methods of improving or enhancing physical performance or recovery from physical performance provided herein comprise: (i) administration of an effective amount of a dosage form comprising a second formulation (“Pro-Sport Formulation”) according to any of the embodiments provided herein to a subject prior to the physical performance and (ii) administration of an effective amount of adosage form of a second formulation (“Active Formulation”) according to any of the embodiments provided herein following the physical performance. In certain embodiments, the dosage form comprising the second formulation is administered to a subject about 15 minutes, 20 minutes, 25 minutes, 30 minutes, 35 minutes, 40 minutes, 45 minutes, 50 minutes, 55 minutes, or 60 minutes before the physical performance. In certain embodiments, the dosage form comprising the second formulation is administered to a subject about 15-60 minutes, 20-60 minutes, or 20-30 minutes before the physical performance. In certain embodiments, administration of the dosage form comprising the second formulation is immediately following the physical performance or about 5 minutes, 10 minutes, 15 minutes, 20 minutes, 25 minutes, 30 minutes, 45 minutes, 50 minutes, 55 minutes, 60 minutes, 70 minutes, 80 minutes, 90 minutes, or 2 hours or more after the physical performance. In certain embodiments, administration of the dosage form comprising the second formulation is spaced at least two hours after administration of the dosage form comprising the second formulation. In certain embodiments, the subject is an adult athlete (e.g., Olympic athlete, professional athlete, amateur athlete). Exemplary combination therapy dosage regimens are set forth in FIG. 9B.

### EXAMPLES

### Example 1

### Performance Enhancement in Mice (after 15 Minutes)

20 male CD-1 mice weighing 24-28 g were divided among two groups. Each group received 0.4 ml of either saline solution or a solution of Formulation A through a stomach feeding tube. Formulation A was administered based on the calculation of 0.2 g of dry substance per 1 kg of body weight with the ratio of components as provided in Table 2.

TABLE 2
Formulation A

| Ingredient | % (w/w) |
|---|---|
| Ammonium succinate | 65% |
| Sodium succinate | 10% |
| Succinic acid | 5% |
| Ascorbic acid | 10% |
| Sodium fumarate | 7% |
| Ammonium phosphate | 3% |

The specified quantity of test formulation 0.2 g per 1 kg meets the pharmacological study requirements of increasing the dose for mice 12-fold relative to the maximum dose proposed for humans [49]. A dry mix of the ingredients of Formulation A was dissolved in 0.89% saline solution to the desired concentration. One animal from each group was placed on a two-track treadmill 15 minutes following administration of either Formulation A or the placebo saline solution. The treadmill was operated at a constant speed of 22 m/min. The animals that received Formulation A ran 1.5-1.8 times longer than the animals that received the placebo.

### Example 2

### Performance Enhancement in Mice (after 25 Minutes)

The same general procedure was followed as described in Example 1. Mice received 0.4 ml of either a solution containing Formulation B or a saline solution 25 minutes prior to being placed on the treadmill.

TABLE 3
Formulation B

| Ingredient | % (w/w) |
|---|---|
| Ammonium succinate | 75% |
| Sodium succinate | 10% |
| Succinic acid | 5% |
| Ammonium-sodium succinate | 5% |
| Ammonium phosphate | 5% |

A dry mix of the ingredients of Formulation B was dissolved in saline solution in an amount sufficient to result in administration of 0.125 g dry weight of Formula B per kilogram of body weight. The animals that received Formulation B ran two times longer, on average, than the animals that received saline solution.

### Example 3

### Performance Enhancement in Mice (after 40 Minutes)

The same general procedure was followed as described in Example 1. Mice received 0.4 ml of either a solution containing Formulation C or a saline solution 40 minutes prior to being placed on the treadmill.

TABLE 4
Formulation C

| Ingredient | % (w/w) |
|---|---|
| Ammonium succinate | 25% |
| Sodium succinate | 20% |
| Succinic acid | 15% |
| Calcium succinate | 5% |
| Sodium fumarate | 20% |
| L-carnitine fumarate | 6% |
| Lycopene | 1% |
| α-tocopherol | 3% |
| Ammonium phosphate | 2% |
| Sodium bicarbonate | 3% |

A dry mix of the ingredients of Formulation C was dissolved in saline solution in an amount sufficient to result in administration of 0.175 g dry weight of Formula C per kilogram of body weight. The animals that received Formulation C ran two times longer, on average, than the animals that received the placebo.

### Example 4

### Performance Enhancement in Mice (after 60 Minutes)

The same general procedure was followed as described in Example 1. Mice received 0.4 ml of either a solution containing Formulation D or a saline solution 60 minutes prior to being placed on the treadmill.

TABLE 5
Formulation D

| Ingredient | % (w/w) |
|---|---|
| Ammonium succinate | 25% |
| Sodium succinate | 15% |
| Succinic acid | 10% |
| Ammonium-sodium succinate | 7% |
| L-carnitine fumarate | 10% |
| Sodium pyruvate | 15% |
| Ascorbic acid | 7% |
| Lycopene | 3% |
| Ammonium phosphate | 3% |
| Sodium bicarbonate | 5% |

A dry mix of the ingredients of Formulation D was dissolved in saline solution in an amount sufficient to result in administration of 0.05 g dry weight of Formulation D per kilogram of body weight. The animals that received Formulation D ran 1.5-2 times longer, on average, than the animals that received the placebo.

### Example 5

### Performance Enhancement in Rats

Male Wistar rats weighing 250-300 g were divided into two groups. One group of ten animals received 1.0 ml of a solution containing Formulation E, while a second group of six animals received 1.0 ml of saline solution. Both solutions were administered through a stomach feeding tube 30 minutes prior to exercise.

TABLE 6
Formulation E

| Ingredients | % (w/w) |
|---|---|
| Ammonium succinate | 40% |
| Succinic acid | 5% |
| Sodium fumarate | 10% |
| L-carnitine fumarate | 20% |
| Ascorbic acid | 9% |
| Ammonium phosphate | 3% |
| Sodium bicarbonate | 3% |

A dry mix of the ingredients of Formulation E was dissolved in saline solution in an amount sufficient to result in administration of 0.09 g dry weight of Formulation E per kilogram of body weight. The dose for rats is six times larger than the dose for humans [49]. A weight equal to 6-6.2% of the rat's body weight was attached to each animal's tail with a soft loop. The rat was then placed into a tall cylindrical glass bath filled with warm water (29-30° C.). The height of the bath was sufficient to prevent the animal from floating atop the water and pushing itself against the bottom of the bath with its tail. A stick was used to push the rats away from the bath walls to prevent them from resting by leaning against the sides. The animals that received the placebo began to sink after 15-18 minutes of swimming. The animals that received Formulation E swam for 30-40 minutes before their first submersion into the water.

### Example 6

### Reducing Acidosis in Rats Under Normal Stress

The same general procedure was followed as described in Example 5. A drop of blood was collected from the tail vein of each male rat before administration of either the placebo, an actoprotector having pure ammonium succinate, or Formulation E to determine the blood's pH, buffer base shift (BBS), and ratio of lactate to pyruvate. The rats were then placed in the water bath for 15 minutes of swimming. After swimming, the blood samples were obtained from their tail veins and tested for pH, buffer base shift, and the ratio of lactate to pyruvate. Data are shown in FIG. 5. Swimming for 15 minutes with a weight equal to approximately 6% of the animal's body weight caused deep acidosis in animals that received the placebo. The post-swim acidosis was substantially reduced when Formulation E was administered prior to swimming (FIG. 5).

### Example 7

### Responses in People not Subjected to any Stress

Formulation F was administered in the form of gelatin capsules to two groups of volunteers at a dose of 15 mg per 1 kg of body weight. The first group was made up of seven male scientists aged 30-35 years, weighing 58-70 kg, and having no history of regular athletic activity. The second group was made up of six former athletes aged 38-45 years, weighing 85-112 kg, each having a Master of Sports in rowing.

TABLE 7
Formulation F

| Ingredient | % (w/w) |
|---|---|
| Ammonium succinate | 62% |
| Sodium succinate | 7% |
| Succinic acid | 7% |
| Sodium fumarate | 7% |
| Sodium pyruvate | 7% |
| Ascorbic acid | 10% |

All volunteers noted a sensation of a blood rush. Members of the first group experienced a rush of blood to the face and scalp, while members of the second group experienced a rush of blood to their muscles. All volunteers reported feeling alert and having a desire to some work. One of the members of the first group, who had suffered a serious head trauma with temporary asphyxiation several years earlier, reported an approximately 20-minute-long rush of blood to the head, especially to the face, as if he had taken nitroglycerin.

### Example 8

### Two Types of Response: Arousal and Falling Asleep

Formulation G was administered to seven female scientists and doctors in the form of a capsule.

TABLE 8
Formulation G

| Ingredients | % (w/w) |
|---|---|
| Ammonium succinate | 75% |
| Sodium succinate | 10% |
| Succinic acid | 5% |
| Ammonium-sodium succinate | 5% |
| Ammonium phosphate | 5% |

The average weight of the volunteers was 70 kg. Five women took a dose of 4 mg per kilogram of body weight. These women reported a sensation of alertness lasting 5-6 hours. Two women were very fatigued after a night shift. One woman took a dose of 8 mg per 1 kg of body weight, and the other woman took a dose of 12 mg per 1 kg of body weight. Both women began to feel sleepy 30 minutes later. The woman who had taken the 12 mg per 1 kg of body weight dose was falling asleep while sitting.

### Example 9

### Improving Work Performance and Enhancing Recovery

Formulation G was administered to a group of volunteer athletes. The volunteers were of various disciplines qualified at the levels of First-Class Athlete and Master of Sport Candidate, including eight male rowers, four divers, five male skaters, seven female skaters, four male decathletes and one female swimmer. Each volunteer took a capsule containing Formulation G at a dose of 12-20 mg per kilogram of body weight 30 minutes prior to their regular training exercises. All the volunteers reported greater ease in working through their regular training exercises or more complete recovery following exercise and an induction of diuresis.

### Example 10

### Delayed Response in Chronically Fatigued Athletes

Formulation G was administered to a group of 19 male hockey players from the same team who had completed a long and exhausting tournament. Each volunteer took Formulation G at a dose of 12-18 mg per kilogram of body weight. After 10-15 minutes, the volunteers reported a sensation of heaviness in their hands and feet, sluggishness, and difficulty completing their regular training exercises. This response lasted about an hour, on average. Subsequently, the volunteers reported a feeling of lightness that lasted 1.5-2 hours. Performance improved noticeably. Accordingly, if the body responds in such a way, then the formulation should be administered 1.5-2 hours before an intense workload.

### Example 11

### Reducing Metabolic Acidosis after Submaximal Exercise and Enhancing Performance and Tolerance to Deep Acidosis Under Maximal Endurance Stress

A group of 18 young female speed skaters participated in a month-long training camp. These women were all qualified as First-Class Athletes or Master of Sport candidates, were coached by the same person, and had an average weight of 59 kg (±4 kg). During the training camp, the athletes periodically engaged in two types of stress tests: a 3-minute standard mixed aerobic-anaerobic cycloergometric exercise with a 300 W output, and two weeks later a maximal endurance step cycloergometric exercise, with each step being 50 W and one minute in duration. The athletes were randomly divided into two equal groups in a blinded study. One group received placebo capsules each containing 300 mg of potato starch, while the other group received capsules each containing 300 mg of Formulation H. Each athlete was assigned a sequential number.

TABLE 9
Formulation H

| Ingredient | % (w/w) |
|---|---|
| Ammonium succinate | 45% |
| Succinic acid | 5% |
| Sodium fumarate | 12% |
| L-carnitine fumarate | 12% |
| Sodium pyruvate | 10% |
| Ascorbic acid | 10% |
| Ammonium phosphate | 3% |
| Sodium bicarbonate | 3% |

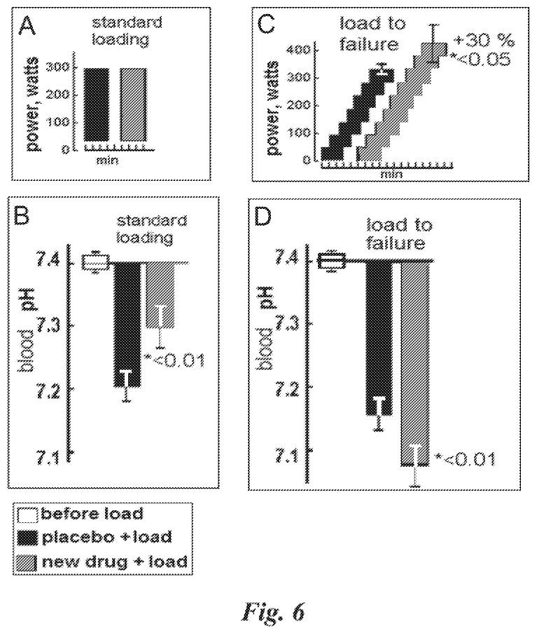

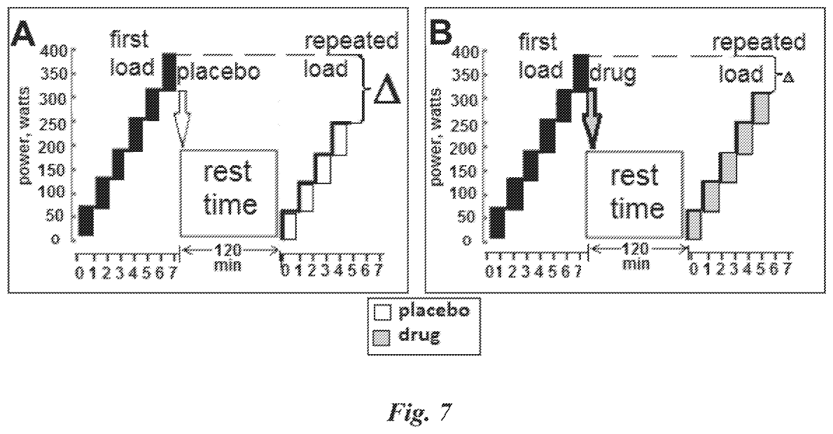

Neither the athletes nor the coach knew which athletes received the placebo and which received Formulation H. The athletes took two capsules, for a total of 600 mg of either placebo or Formulation H, accompanied by half a glass of diluted orange juice, 20 minutes prior to exercising. Before and after exercise, a mixed capillary blood sample was collected from the athletes' fingers to determine the blood's acid-base system parameters. Analysis of the blood sample was performed using the micro Astrup method and clinical acid-base balance analyzer. The “micro-Astrup method” is an interpolation technique for acid-base measurement to determine the base deficit as an expression of metabolic acidosis and the arterial partial pressure of carbon dioxide in the blood as an expression of respiratory acidosis or alkalosis. The base deficit is defined as the amount of strong base that must be added to a liter of fully oxygenated blood to return the pH to 7.40 at a temperature of 37° C. and a pCO₂ of 5.3 kPa. Conversely, the base excess is defined as the amount of strong acid to return the pH to 7.40 under the same conditions. Either a base deficit or a base excess may be referred to as a buffer base shift. A negative value for the base excess can be expressed as a positive value of base deficit. The pH of blood is determined by both a metabolic component and a respiratory component. The respiratory component results from the partial pressure of carbon dioxide in the blood. The metabolic component results primarily from the concentration of bicarbonate in the blood. Statistical analysis of the data showed that the acid pH-shift under an equivalent cycloergometric load (Standard Loading, FIG. 6A) following administration of Formulation H was much smaller (FIG. 6B) than after the placebo. Following administration of Formulation H, the athletes completed a substantially greater workload (FIG. 6C) under a maximal endurance stress despite the development of deeper acidosis (FIG. 6D). Formulation H reduced the acid shift during a fixed-intensity standard submaximal cycloergometric exercise. At the same time, under a maximal endurance stress, administering Formulation H ensured increased tolerance to deeper acidosis and enhanced the ability to complete a greater workload as compared to the group that had initially taken a placebo.

For 2-3 hours after taking Formulation H, the athletes maintained enhanced performance under a fixed submaximal, but not maximal, stress. During a maximal endurance exercise, an increase in performance was observed for 12-15 minutes. Then the individuals transitioned to their regular levels of performance. Furthermore, in athletes who had taken Formulation H, following a period of increased activity, there were no feelings of exhaustion or depleted energy.

### Example 12

### Improving Complete Recovery with a Two-Hour Rest Between Repeated Maximal Endurance Cycloergometric Exercises

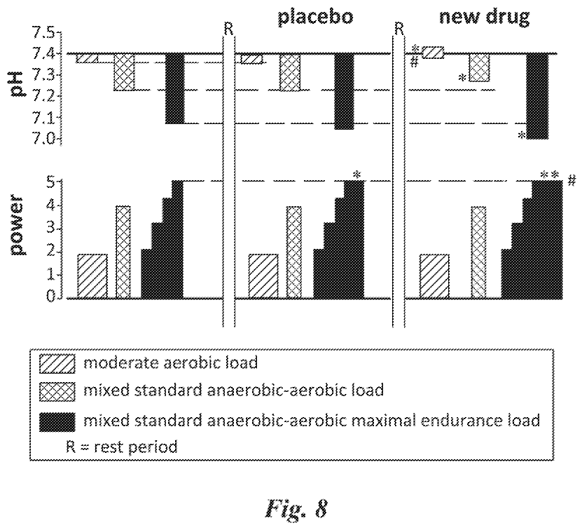

The same general procedure was used as in described Example 11. Ten female athlete volunteers from Example 11 performed step exercises at 60W per step. At the conclusion of the first round of exercise, the athletes were given either a placebo or Formulation H. After a rest period of 120 minutes, the athletes began the second round of exercise. Athletes who received placebo performed two steps less than the step exercise in the first round (FIG. 7A). The athletes who received Formulation H performed one step less than the step exercise in the first round. (FIG. 7B)

### Example 13

### Improving Recovery after Repeated Exercises

A group of 23 female volunteers from a highly qualified speed skating team (First-Class Athletes or Master of Sports Candidates) aged 19-21 years, participating in an intense training program, performed a 10-kilometer run at a heart rate of 120 beats per minute, as a type of restorative therapy. Mixed capillary blood was drawn from the finger of each woman before and after the run to determine the blood's acid-base balance parameters. After the first run, a change in the true pH pointed to a slight tendency toward the development of metabolic acidosis. After the run, 12 randomly selected study participants received 600 mg Formulation H (2 capsules of 300 mg each); two hours later, each of the selected athletes took another 300 mg of Formulation H. The remaining 11 athletes were administered a placebo in the form of potato starch in identical-looking capsules. The day after completing the first run, all athletes ran the same distance at the same pace while their pH was monitored in a similar manner. After the second run, the subgroup of women who had taken the placebo between the two running exercises developed the same insignificant metabolic acidosis: their pH decreased, on average, to 7.34±0.01. The subgroup of women who had taken Formulation H in the interval between the first and second runs demonstrated a tendency toward the development of respiratory alkalosis after the second run. In other words, the blood's acid-base balance attested to a more complete recovery after Formulation H was administered.

### Example 14

### A Short Course of Administering the Agent Improves Exercise Performance

The same group of athlete volunteers from Example 13 was used for this study a week after the running exercises. During this week interval, the athletes did not receive either a placebo or the Formulation H as they engaged in their regular training program The athletes were then divided into two subgroups: 11 athletes were given a placebo and 12 were given the 600 mg Formulation H (2 capsules of 300 mg each) on the day before and the day of each test exercise.

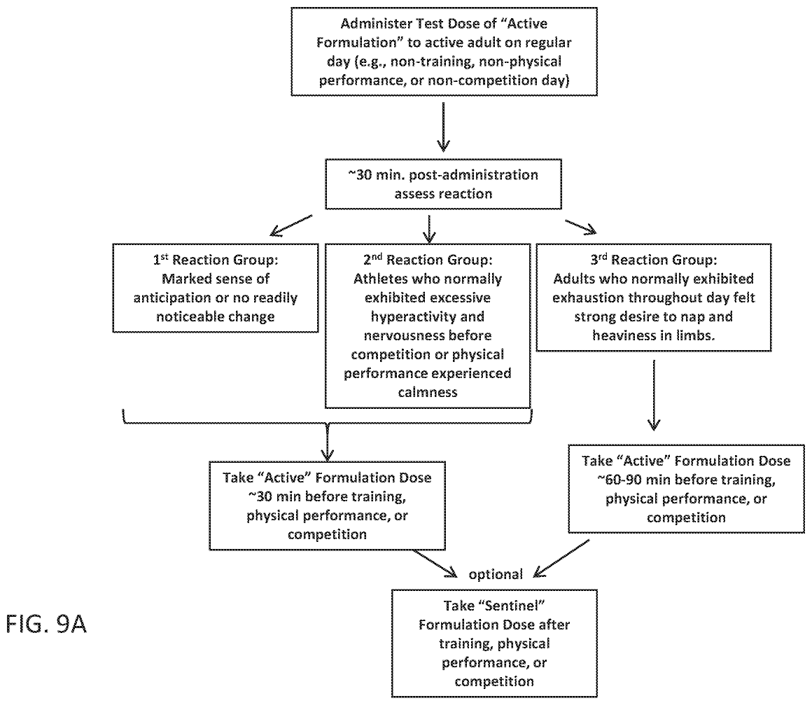

The first exercise was a run of moderate aerobic activity. The athletes who received the placebo developed metabolic acidosis following the run, while the athletes who received Formulation H either developed a slight respiratory alkalosis or had no change in blood pH following the run as compared to a state of rest prior to the exercise. Three days later, the athletes completed a mixed aerobic and anaerobic cycloergometric exercise of fixed intensity and duration, specifically 300 W for four minutes. The athletes who received the placebo again developed metabolic acidosis, while the extent of acidosis was much smaller in those athletes who received Formulation H. A week later, the athletes completed a mixed aerobic and anaerobic maximal endurance cycloergometric exercise. The duration of work and cumulative intensity by the athletes who received Formulation H were greater than that accomplished by the athletes who received the placebo, even though the athletes who received Formulation H showed greater metabolic acidosis following the maximal endurance exercise (FIG. 8). In summary, with the help of a short “course” of Formulation, it was possible to decrease post-exercise acidosis during a standard workload and enhance the athletes' performance and resistance to acidosis during a maximal endurance exercise.

### Example 15

### A Long Course Increases Acidosis Tolerance and Enhances Performance During a Training Program

The same group of female athlete volunteers from Examples 13 and 14 was used for this study. There was a three-month interval between this study and the studies described in Examples 13 and 14. The evaluation took place in winter, when the skaters were in their top athletic shape, over the course of three weeks during their team training. At the beginning of the training, all athletes were tested during a maximal endurance, cycloergometric step exercise. Their blood's acid-base balance parameters were recorded before the exercise and within one minute of its end. The volunteers were then divided into two groups, one of which received 600 mg placebo (two 300 mg capsules) and one of which received 600 mg Formulation H (two 300 mg capsules) every day after breakfast for a period of three weeks. During this time, the athletes continued with their regular training program. At the end of the three weeks, the volunteers again participated in a maximal endurance cycloergometric step exercise, with blood samples taken for testing before and after the exercise. At the end of the three week period, the athletes from the group that received a placebo experienced a one-step increase in their performance compared to their initial state, corresponding to a 7-10% increase in performance, while the pH shift in their blood became about 0.5 deeper. These shifts are significant (p<0.05) per the Wilcoxon signed-rank test [40]. The athletes from the group that received Formulation H experienced a two-step increase in performance and their blood pH level decreased by more than 0.1 (FIGS. 6C and 6D). The differences in gained performance and pH shifts for this group are significant (p<0.01) per the Wilcoxon signed-rank test. The differences in gained performance between the two groups is also significant (p<0.05) per the Mann-Whitney U test [40]. Enhanced performance and the increased ability to continue to perform a maximal endurance exercise with a deeper pH acid shift are characteristic features of a higher level of athletic fitness, which is more representative of the athletes in the “Formulation H” group than of the athletes in the “placebo” group. Therefore, a three-week course of Formulation H during the training program was effective.

### Example 16

### Self-Assessment of the Improvement in Sensory-Motor Responses

A team of 14 male boxers was engaged in a month-long training period. The athletes were randomly divided into two groups, one of which received a placebo and one of which received Formulation H every day for four weeks. Two 300 mg capsules containing either placebo or Formulation H were administered after breakfast at 8:30 am and again after a mid-afternoon meal at 4:30 pm. The athletes' physical performance prior to competition was methodically evaluated. [50] Following the four week group training, the performance of the placebo group increased 19 kg*m/min/kg, while the performance of the group receiving Formulation H increased 22 kg*m/min/kg. The difference in performance between the two groups is significant (p<0.05) per the Mann-Whitney U test.

An important characteristic of martial arts, boxing in particular, is a self-assessment performed by athletes after maximal workloads. The self-assessment of experienced athletes after maximal workloads usually corresponds to objective data, including sensory-motor response indicators. The sensory-motor responses are evaluated by the effectiveness of attacks and the reliability of defense [51]. Following the pre-competition training program, these indicators increased, on average, by 10-15% for the “placebo” group and by 30-40% for the “Formulation H” group (Mann-Whitney nonparametric test p<0.05).

### Example 17

### Impact of a Single 600 Mg Dose on Heart Rate (Treadmill Test)

An athletic male volunteer, 45 years of age, who used to participate in sports and is in good physical shape, participated in treadmill exercises. An Ultimate Treadmill Challenge Company treadmill was programmed to run at a pace that resulted in a gradual increase of the volunteer's heart rate to 145 beats per minute (bpm) and then to maintain that peak heart rate for one minute. The load was then decreased twofold and the corresponding decrease in heart rate was measured following the end of the “peak” load. This exercise was performed ten times: five times with administration of a capsule containing 600 mg Formulation I 20 minutes before exercise and five times without such administration.

TABLE 10
Formulation I

| Ingredient | % (w/w) |
|---|---|
| Ammonium succinate | 55% |
| Succinic acid | 10% |
| Sodium fumarate | 10% |
| Potassium fumarate | 5% |
| Sodium pyruvate | 10% |
| Ammonium phosphate | 3% |
| Sodium bicarbonate | 7% |

Without Formulation I, the volunteer's heart rate decreased to 135 bpm after 30 seconds and to 125 bpm after 90 seconds. When 600 mg of Formulation I was administered 20 minutes before the exercise, following a reduction in the workload, the volunteer's heart rate decreased to 112 bpm after 30 seconds and to 95 bpm after 90 seconds. In addition, the pace required to reach a peak heart rate of 145 bpm was faster after administration of Formulation I than without administration of Formulation I, although the volunteer reported that the workload seemed easier. The change in decrease of heart rate after 90 seconds was significant (t=3.841 and p<0.01).

### Example 18

### Impact of a Single 600 Mg Dose on Heart Rate (Rowing Test)

Two athletes, a 17-year-old First Class athlete and a 22-year-old Master of Sport athlete, participated in rowing exercises. The athletes took either a 600 mg placebo capsule or 600 mg Formulation J capsule 40 minutes before exercise on a rowing machine. The exercise interval lasted eight minutes. The rowing machine resistance was set at the same high level to simulate a maximal competitive load. Each athlete was tested twice with the placebo and twice with a random administration of the agent. Each exercise valuations were spaced one week apart. A Firstbeat Bodyguard heart rate variability device, Version 4.7.3.1, was used to record performance characteristics of the cardiovascular and respiratory systems. It was observed that during the placebo trials, the young First-Class athlete experienced copious sweating and pronounced shortness of breath. During the placebo trials, his pre-exercise heart rate was 60-64 bpm, while the heart rate during the exercise reached 202-208 bpm. With Formulation J treatment, the 17-year-old athlete had a pre-exercise heart rate of 50-55 bpm and a heart rate of 167-170 during exercise. With Formulation J treatment, the 17-year-old athlete experienced less sweating and less pronounced shortness of breath. During the placebo trials, the 22-year-old athlete had a heart rate of 60-63 before the exercise and 182-185 during the exercise. With Formulation J treatment, the athlete's heart rate was 54-57 at rest before the exercise and 147-153 during the exercise. In summary, following treatment with Formulation J, both athletes experienced a decrease in heart rate at rest before the exercise and during the standardized “competition-style” exercise. Treatment with Formulation J led to a faster slowing of the heart rate following the completion of the workload.

TABLE 11
Formulation J

| Ingredient | % (w/w) |
|---|---|
| Ammonium succinate | 60% |
| Calcium succinate | 5% |
| Succinic acid | 10% |
| Potassium fumarate | 5% |
| Sodium pyruvate | 10% |
| Ammonium phosphate | 3% |
| Sodium bicarbonate | 7% |

### Example 19

### Maintenance of Antioxidant Systems

A group of volunteers consisting of 12 men and 15 women aged 54-65 were selected from a fitness center per their trainer's recommendation. The subjects for this group were selected on the basis of their complaints that, after six months of moderate and time-tested exercises, recovery during regular rest periods between their fitness center visits was insufficient. Workout sessions were starting to get missed due to the lack of confidence in their abilities; the usual feeling of post-exercise increase in mood was gone. Massage therapy and recovery sessions in the sauna no longer produced the expected beneficial effect. Blood samples were obtained for testing of biochemical properties including the concentrations of glucose, creatinine, urea and lactate, the lactate/pyruvate ratio, and activities of alanine and aspartate aminotransferase. Blood samples were also tested for hormone levels (follicle-stimulating hormone, corticosterone, and estradiol for women and testosterone for men). There were no significant shifts or deviations from previously obtained data. However, some volunteers experienced a clearly defined tendency toward a reduction in the integral antioxidant activity of the urine, identifiable by means of BioVision colorimetric assay kit. This colorimetric test is based on the reduction of Cu2+ to Cu+ as a chelating colorimetric probe. The absorption peak is registered in the spectrum of 570 nm. The peak's height is proportional to the general antioxidant capacity of the studied liquid.

The volunteers were divided into two groups: a first group of 11 volunteers with normal urine antioxidant activity and a second group of 16 volunteers with antioxidant activity levels 1.5-2 times lower than normal. The volunteers signed an informed consent form, which described the objective of the study and administration of the developed agent. All volunteers were offered a four-week course of the developed agent. Members of the first group were given 300 mg capsules of Formulation K, while members of the second group were given 400 mg capsules of Formulation K. Both groups were instructed to take one capsule daily after breakfast for four weeks.

TABLE 12
Formulation K

| | | wt in 300 | wt in 400 |
|---|---|---|---|
| Ingredient | % (w/w) | mg dose | mg dose |
| Ammonium succinate | 40% | 120 mg | 160 mg |
| Calcium succinate | 10% | 18 mg | 24 mg |
| Sodium succinate | 6% | 18 mg | 24 mg |
| Succinic acid | 5% | 15 mg | 20 mg |
| L-carnitine fumarate | 7% | 21 mg | 28 mg |
| Potassium fumarate | 3% | 9 mg | 12 mg |
| Ascorbic acid | 10% | 30 mg | 40 mg |
| Lycopene | 3% | 9 mg | 12 mg |
| Dihydroquercetin | 6% | 18 mg | 24 mg |
| Vitamin E (α-tocopherol) | 7% | 21 mg | 28 mg |
| Ammonium phosphate | 3% | 9 mg | 12 mg |

The volunteers continued with their regular exercise schedule and intensity. Of the 27 volunteers, 25 completed the four week course and reported a significant improvement in alertness and a decrease in anxiety and self-doubt. The group that had reduced total urine antioxidant activity before administration of Formulation K experienced a nearly complete reestablishment of urine antioxidant activity level at the end of the four-week period.

For the group of older clients, the observed symptoms may to some extent be connected to an overload of the body's own antioxidant systems. A course of Formulation K promoted improved performance, re-appearance of the previously lost desire to exercise, and an improved mood. This was accompanied by the recovery of the endogenous integral activity of the antioxidant system of the urine. In other words, the administration of low doses of the exogenous antioxidants included in Formulation K was sufficiently effective to support the body's own antioxidant systems. The antioxidants administered as part of Formulation K did not suppress, but rather boosted, the diminished antioxidant activity in the studied volunteers.

### Example 20

### A Short Course Improves Tolerance to Stress and Changes in Time Zones

A group of seven female musicians in a string ensemble traveled from Saint Petersburg to Beijing and were offered capsules of Formulation L. Three of the musicians accepted 300 mg capsules containing Formulation L, while the other four musicians declined.

TABLE 13
Formulation L

| Ingredient | % (w/w) |
|---|---|
| Ammonium succinate | 40% |
| Sodium succinate | 15% |
| Succinic acid | 15% |
| Sodium fumarate | 10% |
| Dihydroquercetin | 10% |
| Ammonium phosphate | 5% |
| Sodium bicarbonate | 3% |

Prior to their departure from Saint Petersburg, three of the musicians (including the first violin) took a 300 mg capsule of Formulation L. During the flight, the airplane was unexpectedly rerouted to Helsinki, where it was delayed for 18 hours. The day after the first dose, while in Helsinki, the same three musicians took another 300 mg of Formulation L. From there the flight was directed to Singapore, where it was delayed for 24 hours. The same three women took 600 mg of Formulation L with a meal on the third day after their departure and spent half a day shopping. The other musicians who did not take Formulation L felt tired. Early the next morning the airplane departed Singapore for Beijing, where four hours after their arrival the string ensemble performed a concert. Following the concert, the three musicians who had been taking Formulation L for three days felt alert and “fresh.” The rest of the musicians felt tired and “depleted.”

### Example 21

### Hypnotic Effect

Given the agent's inhibitory and hypnotic effect, a group of volunteers consisting of four men and five women, all suffering from chronic fatigue, took 1 300 mg capsule containing Formulation M before bed. The volunteers reported an improvement in falling asleep and feeling refreshed after sleeping following treatment with Formulation M before bedtime.

TABLE 14
Formulation M

| Ingredient | % (w/w) |
|---|---|
| Ammonium succinate | 42% |
| Sodium succinate | 8% |
| Succinic acid | 8% |
| Potassium fumarate | 17% |
| L-carnitine fumarate | 17% |
| Ammonium phosphate | 8% |

### Example 22

### Aid for Stressful Work Strain and Excessive Fatigue

It was found that a significant portion of a scientific research organization's staff was suffering from pronounced symptoms of chronic strain and fatigue during a period of annual report preparation in addition to regular work. This was manifested as: increased irritability, a marked decrease in performance, miscalculation of effort and time resulting in a time crunch, disruption in communication among staff, anxiety, decreased appetite or the opposite night-time overeating (a clear sign of a stressed state), poor sleep (difficulty falling asleep, early awakening without feeling refreshed). Older staff members experienced higher than usual meteosensitivity and occasional vegetovascular dystonia from unwarranted increases in arterial blood pressure to a lower than usual non-resting blood pressure with a feeling of weakness.

Among 112 staff members who volunteered to take part in the evaluation, 45 employees of both genders were selected. The volunteer selection criteria were the absence of exacerbations of chronic illnesses for two months and the absence of polymorbidity or suspected cancers. The selected volunteers were divided into four groups: two 10-person groups, A and B, composed of younger people aged 40-50 years, and groups C and D composed of 13 and 12 people, respectively, aged 62-70 years. The groups were randomly divided into control and experimental groups. None of the study participants knew what he or she was taking, and everyone was told that they would be taking multivitamins, including small signal doses of metabolic support supplements.

Every morning after breakfast, volunteers from the control groups (A and C) took a multivitamin containing 40% of the daily doses of Vitamins B1, B2, B6, and folic and ascorbic acids. At the same time, volunteers from the experimental groups (B and D) took capsules containing 300 mg of Formulation N:

TABLE 15
Formulation N

| Ingredient | % (w/w) | wt in 300 mg dose |
|---|---|---|
| Ammonium succinate | 42% | 125 mg |
| Succinic acid | 8% | 25 mg |
| Sodium succinate dibase hexahydrate | 8% | 25 mg |
| Potassium fumarate | 17% | 50 mg |
| Ammonium phosphate | 8% | 25 mg |
| L-carnitine fumarate | 17% | 50 mg |

The multivitamin or multivitamin+Formulation N were administered to the control and experimental groups, respectively, for two weeks. Each volunteer was required to keep a log of the aforementioned symptoms. Two physicians examined the volunteers before they began to take the agents (during the selection period), a week later, at the end of the two-week course, and then once again in two weeks.

The volunteers in the younger control group, A, noticed a positive effect, primarily a decrease in irritability and anxiety, only 10-14 days after starting the vitamin complex.

In experimental group B, receiving the complete complex with Formulation N, there was an improvement in well-being in as early as 3-5 days, which was manifested as a relaxed state, improved sleep, increased energy, and enhanced performance. The observed positive effect was sustained, and the sense of well-being continued throughout the entire period of observation, including the two weeks following the administration of the agent.

In control group C, no significant shifts in well-being were registered throughout the entire duration of observation. Moreover, some women observed heaviness in their legs and a certain slowness in performance of their work, which, however, did not affect their general sense of well-being.

All volunteers in experimental group D, without exception, noted an apparent improvement in their sense of well-being after taking the proposed composition 5-7 times. Along with improved sleep, the absence of irritability and anxiety, they noted that their performance improved and that they needed less effort and time to complete their work. At the same time, their appetite normalized and there was no need to overeat in the evening. After the two-week course, all volunteers in this group noted not only an improvement in sleep, but also a restored feeling of “freshness” in the morning after a night's rest. The aforementioned effects were noted throughout the entire observation period, even after the administration of the agent was complete.

Moreover, there was an obvious decrease in meteosensitivity and a nearly complete disappearance of vegetovascular dystonia. Independent monitoring did not show a single volunteer experiencing an increase in heart rate or arterial blood pressure. On the contrary, the heart rate at rest decreased, on average, by 4-7 beats per minute in a sitting position.

The volunteers' leukocyte differential counts were analyzed prior to the start of the study, and 39 out of 45 volunteers had low lymphocyte/neutrophil ratios and low lymphocyte concentrations of 22-26% given relatively normal general leukocyte levels, which is characteristic of stressed states. This shift was experienced by all volunteers without exception only in experimental groups B and D. The lymphocyte/neutrophil ratio increased. The percentage of lymphocytes in some volunteers increased to 35-40%. Such shifts in animals and humans indicate the absence of chronic stress.

### Example 23

### Enhancing Power Exercise Performance

Mixed martial arts athletes perform power exercises to perfect strikes. The core component of this power training is that each athlete must strike a truck tire with a 9.5 kg club 50 times with each hand. The height of a lying tire is 35-40 cm. The strikes must bend the tire. Under such stress, the athletes train to complete exhaustion to the point where one out of the five lost consciousness fairly often at the end of the exercise. The coach offered the martial artists to take Formulation O before training. Each athlete, weighing about 80-90 kg, was given four sets Formulation O based on the calculation of three capsules per training session. The athletes took Formulation O 30 minutes before the start of the impact power exercise with the club. The 325 mg formulation is set forth in Table 15.

TABLE 16
Formulation O

| | | wt in |
|---|---|---|
| Ingredient | % (w/w) | 325 mg dose |
| Ammonium succinate | 77% | 250 mg |
| Sodium succinate dibase hexahydrate | 15% | 50 mg |
| Ascorbic acid (microencapsulated) | 8% | 25 mg |

All five fighters noted a greater ease in performing the strike exercises with a prior administration of the stated composition. They did not collapse from exhaustion at the end of the training. The condition and behavior of the athlete who occasionally exhibited loss of consciousness at the end of the power exercises did not differ from that of his fellow athletes.

### Example 24

### Improvement of Overall Well-being, Memory, Sleep and Coordination in Older People

A group of 16 volunteers, aged 65-78, was selected among retired professional drivers and researchers continuing their scientific pursuits.

Ultrasound studies revealed various degrees of hepatic steatosis (more pronounced in the retired drivers), combined with slow performance on a number connection test (NCT). In addition, after waking up in the morning and starting to engage in physical activity, a number of the group's members experienced a sudden irregularity in heart rate without any significant deviations in blood pressure. According to a heart rate monitor, their heart rates increased by 40-50% from the initial rate (in a lying-down or sitting position), even to the point of feeling weak. The cessation of physical activity and transition to a lying-down position led to normalization of heart rate. In approximately one hour, a gradual increase in physical activity was not accompanied by sudden heart rate fluctuations, and these essentially did not recur throughout the day. The volunteers with more pronounced hepatic steatosis complained of having a “weary head” for 1-2 hours after waking up in the morning.

At the same time, there was observable neuromuscular incoordination presenting as an inability to trace a given shape and as the emergence of a slight tremor during a writing exercise. Moreover, these phenomena were exacerbated when the subject “tried harder” to write more clearly and neatly, especially when filling out official forms.

Certain members of the group, who had a steady gait in the morning following a night's sleep and throughout the first half of a day, exhibited slight deviations from a straight-line trajectory when walking (inability to walk in a straight line), specifically when they became tired in the evening, without any signs of dizziness either at rest or while walking.

Signs of irritability appeared, sometimes to the point of nonverbal aggression. At the same time, the volunteers were very upset that they were slightly distracted from the task at hand. A more or less strong external irritant could cause a shift of attention to such an extent that, for some time, the person became distracted and forgot what activity he or she was performing or was about to perform.

In addition, operational memory deteriorated more in the retired drivers than in the researchers. Moreover, with age, there was greater difficulty in acquiring new skills, specifically in using navigation systems on new electronic devices.

At the same time, a significant proportion of volunteers noted frequently experiencing cold extremities (especially feet) while maintaining a regular pulse around their ankles.

Naturally, these phenomena reflect the presence not only of some hepatic encephalopathy, but also of dynamic and organic disorders of the central and peripheral blood flow and, possibly, the onset of early myocardiodystrophy.

In selecting volunteers, the same exclusion criteria specified in Example 22 were used. Subjects with a history of exacerbations of chronic pancreatitis in the past six months were also excluded.

Prior to the study described below, all members of the group were taking high doses of various antioxidants as supplements or medication for 2-3 months. The lack of noticeable improvement in well-being or of alleviation of the aforementioned symptoms led them to volunteer at the research center, where they were offered 315 mg of Formulation P, certified for safety by an accredited international laboratory for biological studies.

TABLE 17
Formulation P

| | | wt in |
|---|---|---|
| Ingredient | % (w/w) | 315 mg dose |
| Ammonium succinate | 47.6% | 150 mg |
| Succinic acid (natural conformer) | 5.4% | 17 mg |
| Sodium succinate dibase hexahydrate | 9.5% | 30 mg |
| Potassium fumarate | 6.3% | 20 mg |
| Ammonium phosphate | 2.5% | 8 mg |
| L-carnitine fumarate | 11.1% | 35 mg |
| Lycopene | 1.9% | 6 mg |
| Dihydroquercetin | 5.4% | 17 mg |
| α-tocopherol (microencapsulated) | 5.4% | 17 mg |
| Ascorbic acid (microencapsulated) | 4.8% | 15 mg |

Formulation P was distributed in either three-layer dry packets or gelatin capsules.

For three weeks, the volunteers took the agent daily after breakfast, followed by tea, water, juice, or a sour-milk product. The volunteers subjectively and independently assessed the efficacy of the agent. They performed movement coordination and number connection tests in an ambulatory setting under specialist supervision. The efficacy assessment was performed upon the completion of the three-week course and again two weeks later. No operational memory test was performed.

None of the volunteers noted any negative symptoms, the development of which could have been attributed to the administration of the developed agent. An improvement of the following symptoms was noted in order of decreasing frequency, and, conversely, at a later time than the start of the treatment.

1. After 2-3 days, sleep improved to a point where falling asleep did not require sedatives or alcohol, as had been the practice prior to the treatment.

2. Sleep was refreshing even when it did not last for more than six hours. In the morning, the feeling of “cloudiness” or a “weary head” was less pronounced to a point where this symptom was no longer reported after 5-8 days.

3. Signs of morning heart rate irregularity disappeared after essentially one week, and heart rate decreased overall under static and dynamic loads, as well as during an orthostatic test.

4. Cold hands and feet in the evening were observed less frequently.

5. There was a feeling of lightness and enhanced performance under regular physical and mental loads.

6. Irritability and anxiety decreased, unwarranted aggression manifested itself less frequently, communication with family and coworkers improved.

7. The ability to focus one's attention improved significantly, while distractibility during work decreased.

8. At the end of the study, a faster completion of the NCT was detected. If prior to the treatment the NCT duration reached 60-75 s instead of the normal 30-35 s, then after the treatment the NCT time decreased to 40-45 s. At the same time, a more precise tracing of shapes was observed. Half of the volunteers did not exhibit shaky handwriting when filling out forms; wavering from side to side was noticed only in the evening.

9. Working with a smartphone became easier.

In addition to the above, the present disclosure provides the following enumerated embodiments.

### Embodiment 1

A dosage form for oral administration, comprising a formulation comprising:
about 24%-36% by weight ammonium succinate;
about 4%-16% by weight sodium succinate;
about 4%-12% by weight succinic acid;
about 8%-16% by weight potassium fumarate or sodium fumarate;
about 2%-8% by weight ammonium phosphate; and
about 8%-16% by weight L-carnitine fumarate or a mixture of L-carnitine and sodium fumarate at a 1:1 ratio.

### Embodiment 2

The dosage form of embodiment 1, wherein the formulation comprises 8%-16% potassium fumarate.

### Embodiment 3

The dosage form of embodiment 1 or 2, wherein the formulation comprises 8%-16% L-carnitine fumarate.

### Embodiment 4

The dosage form of any one of embodiments 1-3, wherein sodium succinate is sodium succinate dibase hexahydrate.

### Embodiment 5

The dosage form of any one of embodiments 1-4, wherein one or more components of the formulation is microencapsulated.

### Embodiment 6

The dosage form of any one of embodiments 1-5, wherein the formulation further comprises a pharmaceutical excipient.

### Embodiment 7

The dosage form of embodiment 6, wherein the excipient comprises a filler, a lubricant, an anticaking agent, or any combination thereof.

### Embodiment 8

The dosage form of embodiment 7, wherein the formulation comprises filler in an amount of about 10%-20% by weight.

### Embodiment 9

The dosage form of embodiment 7 or 8, wherein the filler comprises microcrystalline cellulose.

### Embodiment 10

The dosage form of any one of embodiments 7-9, wherein formulation comprises lubricant in an amount of about 1%-5% by weight.

### Embodiment 11

The dosage form of any one of embodiments 7-10, wherein the lubricant comprises magnesium stearate.

### Embodiment 12

The dosage form of any one of embodiments 7-11, wherein the formulation comprises anticaking agent in an amount of about 1%-5% by weight.

### Embodiment 13

The dosage form of any one of embodiments 7-12, wherein the anticaking agent comprises silicon dioxide.

### Embodiment 14

The dosage form of any one of embodiments 1-13, wherein the ratios of ammonium succinate, sodium succinate, succinic acid, potassium fumarate or sodium fumarate, ammonium phosphate, and L-carnitine fumarate or a mixture of L-carnitine and sodium fumarate at a 1:1 ratio are about 5:1:1:2:1:2, respectively.

### Embodiment 15

The dosage form of any one of embodiments 1-14, wherein the dosage form is a capsule, tablet, powder, or liquid.

### Embodiment 16

The dosage form of any one of embodiments 1-15, wherein the amount of formulation in the dosage form is about 150 mg 1,700 mg.

### Embodiment 17

The dosage form of embodiment 16, wherein the amount of formulation in the dosage form is about 250 mg 600 mg.

### Embodiment 18

The dosage form of embodiment 16, wherein the amount of formulation in the dosage form is about 300 mg 500 mg.

### Embodiment 19

The dosage form of embodiment 16, wherein the amount of formulation in the dosage form is about 350 mg 400 mg.

### Embodiment 20

The dosage form of embodiment 16, wherein the amount of formulation in the dosage form is about 375 mg.

### Embodiment 21

The dosage form of any one of embodiments 15-20, wherein the dosage form is a capsule and further comprises a capsule shell.

### Embodiment 22

The dosage form of embodiment 21, wherein the capsule shell comprises hydroxypropyl methylcellulose.

### Embodiment 23

The dosage form of any one of embodiments 15-20, wherein the dosage form is a tablet.

### Embodiment 24

The dosage form of embodiment 23, wherein the tablet is coated to 1%-10% weight gain with a film coating.

### Embodiment 25

The dosage form of embodiment 24, wherein the film coating is a water resistant coating.

### Embodiment 26

A method of enhancing physical performance or recovery following physical performance in a subject, comprising orally administering an effective amount of the dosage form of any one of embodiments 1-25 to the subject.

### Embodiment 27

The method according to embodiment 26, wherein the dosage form is administered to the subject at about 3-25 mg/kg.

### Embodiment 28

The method according to embodiment 26, wherein the dosage form is administered to the subject at about 5-20 mg/kg.

### Embodiment 29

The method according to embodiment 26, wherein the dosage form is administered to the subject at about 8-18 mg/kg.

### Embodiment 30

The method according to embodiment 29, wherein the dosage form is administered to the subject about 12-18 mg/kg.

### Embodiment 31

The method according to any one of embodiments 26-30, wherein the dosage forms is administered to the subject about one to three times per day.

### Embodiment 32

The method according to any one of embodiments 26-31, wherein the dosage form is administered to the subject for an initial treatment period of about 2-60 consecutive days.

### Embodiment 33

The method according to any one of embodiments 26-32, wherein the dosage form is administered to the subject for an initial treatment period of about 2-7 consecutive days.

### Embodiment 34

The method according to any one of embodiments 26-32, wherein the dosage form is administered to the subject for an initial treatment period of about 14-28 consecutive days.

### Embodiment 35

The method according to any one of embodiments 26-32, wherein the dosage form is administered to the subject for an initial treatment period of about 30-60 consecutive days.

### Embodiment 36

The method according to any one of embodiments 32-35, wherein following the initial treatment period is a period of about 2-14 consecutive days in which the dosage form is not administered to the subject before resuming treatment with the dosage form.

### Embodiment 37

The method according to any one of embodiments 32-36, wherein following an initial treatment period is a period of about 10-14 consecutive days in which the dosage form is not administered to the subject before resuming treatment with the dosage form.

### Embodiment 38

The method according to embodiment 32, wherein following an initial treatment period of about 60 consecutive days is a period of about 14 consecutive days in which the dosage form is not administered to the subject before resuming treatment with the dosage form.

### Embodiment 39

The method according to any one of embodiments 32-38, wherein the dosage form is administered to the subject with or immediately following a meal.

### Embodiment 40

The method according to any one of embodiments 32-39, wherein the dosage form is administered to the subject about 15-90 minutes before exercise.

### Embodiment 41

The method according to any one of embodiments 32-38, wherein the dosage form is administered to the subject after exercise.

### Embodiment 42

The method according to any one of embodiments 26-41, wherein the subject is human.

### Embodiment 43

The method according to any one of embodiments 26-42, wherein the subject is an adult.

### Embodiment 44

The method according to any one of embodiments 26-43, wherein the subject is an athlete.

### Embodiment 45

The method according to embodiment 42, wherein the subject is about 18-80 years old.

### Embodiment 46

The method according to embodiment 45, wherein the subject is about 18-65 years old.

### Embodiment 47

The method according to embodiment 45, wherein the subject is about 18-50 years old.

### Embodiment 48

The method according to any one of embodiments 26-44, wherein the subject is about 18-40 years old.

### Embodiment 49

The unit dosage form of any one of embodiments 1-25 for use in enhancing physical performance or recovery following physical performance in a subject.

### Embodiment 50

A dosage form for oral administration, comprising a formulation comprising:
about 54%-76% by weight ammonium succinate;
about 6%-14% by weight sodium succinate; and
about 6%-14% by weight ascorbic acid.

### Embodiment 51

The dosage form of any one of embodiment 50, wherein sodium succinate is sodium succinate dibase hexahydrate.

### Embodiment 52

The dosage form of embodiment 52, wherein one or more components of the formulation is microencapsulated.

### Embodiment 53

The dosage form of embodiment 52, wherein the component that is microencapsulated is ascorbic acid, ammonium succinate, or both.

### Embodiment 54

The dosage form of embodiment 52 or 53, wherein the component is microencapsulated with palm oil, soybean oil, cottonseed oil, sunflower oil, or coconut oil.

### Embodiment 55

The dosage form of any one of embodiment 50-54, wherein the formulation further comprises a pharmaceutical excipient.

### Embodiment 56

The dosage form of embodiment 55, wherein the excipient comprises a lubricant, an anticaking agent, or both.

### Embodiment 57

The dosage form of embodiment 56, wherein the formulation comprises lubricant in an amount of about 1%-5% by weight.

### Embodiment 58

The unit dosage form of embodiment 56 or 57, wherein the lubricant is magnesium stearate.

### Embodiment 59

The dosage form of any one of embodiments 56-58, wherein the formulation comprises anticaking agent in an amount of about 1%-6% by weight.

### Embodiment 60

The dosage form of any one of embodiments 56-59, wherein the anticaking agent is silicon dioxide.

### Embodiment 61

The dosage form of any one of embodiments 50-60, wherein the ratios of ammonium succinate, sodium succinate, and ascorbic acid are at about 10:2:1, respectively.

### Embodiment 62

The dosage form of any one of embodiments 50-61, wherein the dosage form is a capsule, tablet, powder, or liquid.

### Embodiment 63

The dosage form of any one of embodiments 50-62, wherein the amount of formulation in the dosage form is about 150 mg 1,700 mg.

### Embodiment 64

The dosage form of embodiment 63, wherein the amount of formulation in the dosage form is about 250 mg 600 mg.

### Embodiment 65

The dosage form of embodiment 63, wherein the amount of formulation in the dosage form is about 300 mg 500 mg.

### Embodiment 66

The dosage form of embodiment 63, wherein the amount of formulation in the dosage form is about 350 mg 400 mg.

### Embodiment 67

The dosage form of embodiment 63, wherein the amount of formulation in the dosage form is about 360 mg.

### Embodiment 68

The dosage form of any one of embodiments 62-67, wherein the dosage form is a capsule and further comprises a capsule shell.

### Embodiment 69

The dosage form of embodiment 68, wherein the capsule shell comprises hydroxypropyl methylcellulose.

### Embodiment 70

The dosage form of embodiment 62-67, wherein the dosage form is a tablet.

### Embodiment 71

The dosage form of embodiment 70, wherein the tablet is coated to 1%-10% weight gain with a film coating.

### Embodiment 72

The dosage form of embodiment 71, wherein the film coating is a water resistant coating.

### Embodiment 73

A method of enhancing physical performance or recovery following physical performance in a subject comprising orally administering an effective amount of a dosage form of any one of embodiments 50-72 to the subject.

### Embodiment 74

The method according to embodiment 73, wherein the dosage form is administered to the subject at about 3-25 mg/kg.

### Embodiment 75

The method according to embodiment 74, wherein the dosage form is administered to the subject at about 5-20 mg/kg.

### Embodiment 76

The method according to embodiment 74, wherein the dosage form is administered to the subject at about 8-18 mg/kg.

### Embodiment 77

The method according to embodiment 74, wherein the dosage form is administered to the subject at about 12-18 mg/kg.

### Embodiment 78

The method according to any one of embodiments 73-77, wherein the dosage form is administered to the subject about one to about three times per day.

### Embodiment 79

The method according to any one of embodiments 73-78, wherein the dosage form is administered to the subject for an initial treatment period of about 2-60 consecutive days.

### Embodiment 80

The method according to embodiment 79, wherein the dosage form is administered to the subject for an initial treatment period of about 2-7 consecutive days.

### Embodiment 81

The method according to embodiment 79, wherein the dosage form is administered to the subject for an initial treatment period of about 14-28 consecutive days.

### Embodiment 82

The method according to embodiment 79, wherein the dosage form is administered to the subject for an initial treatment period of about 30-60 consecutive days.

### Embodiment 83

The method according to any one of claims 79-82, wherein following the initial treatment period is a period of about 2-14 consecutive days in which the dosage form is not administered to the subject before resuming treatment with the dosage form.

### Embodiment 84

The method according to any one of embodiments 79-82, wherein following the initial treatment period is a period of about 10-14 days in which the dosage form is not administered to the subject before resuming treatment with the dosage form.

### Embodiment 85

The method according to embodiment 79, wherein following an initial treatment period of about 60 consecutive days is a period of about 14 consecutive days in which the dosage form is not administered to the subject before resuming treatment with the dosage form.

### Embodiment 86

The method according to any one of embodiments 73-85, wherein the dosage form is administered to the subject with or immediately following a meal.

### Embodiment 87

The method according to any one of embodiments 73-86, wherein the dosage form is administered to the subject about 15-90 minutes before exercise.

### Embodiment 88

The method according to embodiment 87, further comprising administering to the subject the dosage form of any one of embodiments 1-25 after exercise.

### Embodiment 89

The method according to embodiment 88, wherein an effective amount of the dosage form of any one of embodiments 1-25 is administered to the subject at least two hours after the dosage form of any one of embodiments 50-72 is administered.

### Embodiment 90

The method of any one of embodiments 73-89, wherein the subject is human.

### Embodiment 91

The method of any one of embodiments 73-90, wherein the subject is an adult.

### Embodiment 92

The method according to of embodiment 90, wherein the subject is an athlete.

### Embodiment 93

The method according to embodiment 90, wherein the subject is about 18-65 years old.

### Embodiment 94

The method according to embodiment 90, wherein the subject is about 18-45 years old.

### Embodiment 95

A dosage form for oral administration, comprising a formulation comprising:
about 36%-48% by weight ammonium succinate;
about 7%-14% by weight sodium succinate;
about 4%-9% by weight succinic acid;
about 4%-7% by weight potassium fumarate or sodium fumarate;
about 2%-4% by weight ammonium phosphate;
about 8%-14% by weight L-carnitine fumarate or a mixture of L-carnitine and sodium fumarate at a 1:1 ratio;
about 1%-3% by weight antioxidant;
about 4%-10% by weight dihydroquercetin;
about 4%-10% by weight α-tocopherol; and
about 4%-14% by weight ascorbic acid.

### Embodiment 96

The dosage form of embodiment 95, wherein the antioxidant is curcumin or lycopene.

### Embodiment 97

The dosage form of embodiment 96, wherein the antioxidant is curcumin.

### Embodiment 98

The dosage form of embodiment 96, wherein the antioxidant is curcumin, demethoxycurcumin, bisdemethoxycurcumin, or a combination thereof.

### Embodiment 99

The dosage form of embodiment 96, wherein the antioxidant is lycopene.

### Embodiment 100

The dosage form of embodiment 99, wherein the lycopene comprises lycopene-13-cis-isomer.

### Embodiment 101

The dosage form of any one of embodiments 95-100, wherein sodium succinate is sodium succinate dibase hexahydrate.

### Embodiment 102

The dosage form of any one of embodiments 95-101, wherein one or more components of the formulation is microencapsulated.

### Embodiment 103

The dosage form of embodiment 102, wherein the component that is microencapsulated is α-tocopherol.

### Embodiment 104

The dosage form of embodiment 102 or 103, wherein the component that is microencapsulated is ascorbic acid.

### Embodiment 105

The dosage form of any one of embodiments 102-104, wherein the component that is microencapsulated is curcumin.

### Embodiment 106

The dosage form of embodiment 102, wherein α-tocopherol, ascorbic acid, and curcumin are microencapsulated together or separately.

### Embodiment 107

The dosage form of any one of embodiments 102-106, wherein the component is microencapsulated with palm oil, soybean oil, cottonseed oil, sunflower oil, or coconut oil.

### Embodiment 108

The dosage form of any one of embodiments 95-107, wherein the formulation further comprises a pharmaceutical excipient.

### Embodiment 109

The dosage form of embodiment 108, wherein the excipient comprises a lubricant, an anticaking agent, or both.

### Embodiment 110

The dosage form of embodiment 109, wherein the formulation comprises lubricant in an amount of about 0.1%-5% by weight.

### Embodiment 111

The dosage form of embodiment 109 or 110, wherein the lubricant is magnesium stearate.

### Embodiment 112

The dosage form of any one of embodiments 109-111, wherein the formulation comprises anticaking agent in an amount of about 2-4% by weight.

### Embodiment 113

The dosage form of any one of embodiments 109-112, wherein the anticaking agent is silicon dioxide.

### Embodiment 114

The dosage form of any one of embodiments 95-113, wherein the ratios of ammonium succinate, sodium succinate, succinic acid, potassium fumarate or sodium fumarate, ammonium phosphate, L-carnitine fumarate or a mixture of L-carnitine and sodium fumarate in a 1:1 ratio, antioxidant, dihydroquercetin, α-tocopherol, and ascorbic acid are 10:2:1.13:1.33:0.53:2.33:0.4:1.13:1.13:1, respectively.

### Embodiment 115

The dosage form of any one of embodiments 95-114, wherein the dosage form is a capsule, tablet, powder, or liquid.

### Embodiment 116

The dosage form of any one of embodiments 95-115, wherein the amount of formulation in the dosage form is about 150 mg 1,700 mg.

### Embodiment 117

The dosage form of embodiment 116, wherein the amount of formulation in the dosage form is about 250 mg 600 mg.

### Embodiment 118

The dosage form of embodiment 116, wherein amount of formulation in the dosage form is about 300 mg 500 mg.

### Embodiment 119

The dosage form of embodiment 116, wherein the amount of formulation in the dosage form is about 350 mg 400 mg.

### Embodiment 120

The dosage form embodiment 116, wherein the amount of formulation in the dosage form is about 350 mg.

### Embodiment 121

The dosage form of any one of embodiments 115-120, wherein the dosage form is a capsule and further comprises a capsule shell.

### Embodiment 122

The dosage form of embodiment 121, wherein the capsule shell comprises hydroxypropyl methylcellulose.

### Embodiment 123

The dosage form of any one of embodiments 115-120, wherein the dosage form is a tablet.

### Embodiment 124

The dosage form of embodiment 123, wherein the table is coated to 1-10% weight gain with a film coating.

### Embodiment 125

The dosage form of embodiment 124, wherein the film coating is a water resistant coating.

### Embodiment 126

A method for enhancing physical performance, recovery following physical performance in a subject comprising orally administering an effective amount of a dosage form of any one of embodiments 95-125 to the subject.

### Embodiment 127

The method according to embodiment 126, wherein the dosage form is administered to the subject at about 3-25 mg/kg.

### Embodiment 128

The method according to embodiment 127, wherein the dosage form is administered to the subject at about 5-20 mg/kg.

### Embodiment 129

The method according to embodiment 127, wherein the dosage form is administered to the subject at about 8-18 mg/kg.

### Embodiment 130

The method according to embodiment 127, wherein the dosage form is administered to the subject at about 12-18 mg/kg.

### Embodiment 131

The method according to any one of embodiments 126-130, wherein the dosage forms is administered to the subject about one to about three times per day.

### Embodiment 132

The method according to any one of embodiments 126-131, wherein the dosage form is administered to the subject for an initial treatment period of about 2-60 consecutive days.

### Embodiment 133

The method according to embodiment 132, wherein the dosage form is administered to the subject for an initial treatment period of about 2-7 consecutive days.

### Embodiment 134

The method according to embodiment 132, wherein the dosage form is administered to the subject for an initial treatment period of about 14-28 consecutive days.

### Embodiment 135

The method according to embodiment 132, wherein the dosage form is administered to the subject for an initial treatment period of about 30-60 consecutive days.

### Embodiment 136

The method according to any one of embodiments 132-135, wherein the initial treatment period is followed by a period of about 2-14 consecutive days in which the dosage form is not administered to the subject before resuming treatment with the dosage form.

### Embodiment 137

The method according to any one of embodiments 132-135, wherein the initial treatment period is followed by a period of about 10-14 consecutive days in which the dosage form is not administered to the subject before resuming treatment with the dosage form.

### Embodiment 138

The method according to embodiment 132, wherein the dosage form is administered for an initial treatment period of about 60 consecutive days followed by a period of about 14 consecutive days in which the dosage form is not administered to the subject before resuming treatment with the dosage form.

### Embodiment 139

The method according to any one of embodiments 126-138, wherein the dosage form is administered to the subject with or immediately following a meal.

### Embodiment 140

The method according to any one of embodiments 126-139, wherein the dosage form is administered to the subject after exercise.

### Embodiment 141

The method according to embodiment 140, further comprising administering to the subject an effective amount of the dosage form of any one of embodiments 1-25 about 15-90 minutes before exercise.

### Embodiment 142

The method according to embodiment 141, wherein the dosage form of any one of embodiments 95-125 is administered to the subject at least two hours after the dosage form of any one of embodiments 1-25 is administered.

### Embodiment 143

The method according to any one of embodiments 126-142, wherein the subject is an active adult.

### Embodiment 144

The method according to any one of embodiments 126-143, wherein the subject is human.

### Embodiment 145

The method according to embodiment 144, wherein the subject is about 50-80 years old.

### Embodiment 146

The method according to embodiment 145, wherein the subject is about 50-75 years old.

### Embodiment 147

An agent comprising sodium fumarate, L-carnitine fumarate, sodium pyruvate, succinic acid, sodium succinate, sodium-ammonium succinate, ammonium succinate, ammonium phosphate and sodium bicarbonate, intended for enhancing performance and more complete recovery after intensive loads, and a method for use of said agent which includes the methodology to select conditions for a single oral administration and a course of oral administration.

### Embodiment 148

The agent according to Embodiment 147, characterized in that the fraction of active substances is 0 to 20% for sodium fumarate, 0 to 25% for L-carnitine fumarate, 0 to 15% for sodium pyruvate, 5 to 15% for succinic acid, 10 to 20% for succinate sodium, 20 to 75% for ammonium succinate and 0 to 10% for sodium-ammonium succinate.

### Embodiment 149

The agent according to Embodiments 147 or 148, characterized in that the content of auxiliary substances is 3 to 10% for ammonium phosphate and 0 to 7% for sodium bicarbonate.

### Embodiment 150

The agent according to Embodiments 147, 148, or 149, characterized in that the total content of the components in a single dose is 150-1200 mg.

### Embodiment 151

The method for use of said agent according to any one of Embodiments 147-150, characterized in that the testing of the type of the response of the user's body to the administration of the proposed agent in normal conditions without additional workloads is initially carried out.

### Embodiment 152

The method for use of said agent according to any one of Embodiments 147-151, characterized in that the agent is administered in a single dose 15-60 minutes before the load, in order to enhance performance before intensive work.

### Embodiment 153

The method for use of said agent according to any one of Embodiments 147-152, characterized in that in order to enhance performance during the day, the agent is administered in the morning after a meal.

### Embodiment 154

The method for use of said agent according to any one of Embodiments 147-153, characterized in that in order to make recovery more complete, the agent is administered after the load has ended.

### Embodiment 155

The method for use of said agent according to any one of Embodiments 147-154, characterized in that in order to improve sleep and make nighttime recovery more complete, the agent is administered 2-3 hours before sleep.

### Embodiment 156

The method for use of said agent according to any one of Embodiments 147-155, characterized in that in order to achieve a stable enhancement in performance and make recovery after loads more complete, the drug is administered in a course over 2 to 4 weeks.

### Embodiment 157

An agent comprising succinic acid, sodium succinate, ammonium-sodium succinate, calcium succinate, ammonium succinate, sodium fumarate, potassium fumarate, L-carnitine fumarate, sodium pyruvate, ammonium phosphate, sodium bicarbonate, ascorbic acid, dihydroquercetin, lycopene and alpha-tocopherol and intended to be administered internally either as a single dose or as a course for the purpose of enhancing performance and enabling a more complete recovery after recurrent stresses.

### Embodiment 158

The agent of Embodiment 157, wherein the natural metabolites of tissue metabolism include between 0 and 15% of various conformers of succinic acid, between 0 and 20% of sodium succinate, between 0 and 10% of calcium succinate, between 0 and 75% of ammonium succinate, between 0 and 10% of ammonium-sodium succinate, between 0 and 20% of sodium fumarate, between 0 and 5% of potassium fumarate, between 0 and 20% of L-carnitine fumarate, and between 0 and 15% of sodium pyruvate.

### Embodiment 159

The agent in Embodiments 157 or 158, wherein the water-soluble and fat-soluble natural antioxidants include between 0 and 10% of ascorbic acid, between 0 and 10% of dihydroquercetin, between 0 and 3% of lycopene, and between 0 and 7% of alpha-tocopherol.

### Embodiment 160

The agent in any one of Embodiments 157-159, wherein the natural buffers include between 0 and 5% of diammonium phosphate and between 0 and 7% of sodium bicarbonate.

### Embodiment 161

The agent in any one of Embodiments 157-160, wherein the total content of components in a single dose is 150-1,700 mg, which corresponds to 3-20 mg of the agent per 1 kg of human body weight.

### Embodiment 162

A method for use of the agent in any one of Embodiments 157-161, wherein there is first a test of the user's reaction to administering the agent under normal conditions without additional workloads.

### Embodiment 163

The method of use of the agent in any one of Embodiments 157-162, wherein a single dose of the agent is administered 15-60 minutes before an intense workload to enhance performance.

### Embodiment 164

The method for use of the agent in any one of Embodiments 157-163, wherein the agent is administered in the first half of the day to enhance performance throughout the day.

### Embodiment 165

The method for use of the agent in any one of Embodiments 157-164, wherein the agent is administered after a workload is completed to enhance recovery from fatigue.

### Embodiment 166

The method for use of the agent in any one of Embodiments 157-165, wherein the agent is administered before bedtime to facilitate falling asleep and to improve the quality of sleep.

### Embodiment 167

The method for use of the agent in any one of Embodiments 157-166, wherein the agent is administered as a 3-28 day course to enable more sustainable performance enhancement and a more complete recovery after stresses.

### Embodiment 168

The method for use of the agent in any one of Embodiments 157-167, wherein the agent is administered as a course during a period of stress to enable more effective performance enhancement and a more complete recovery after stresses.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 62/678,124 filed May 30, 2018, and U.S. Provisional Patent Application No. 62/586,665 filed Nov. 15, 2017, are incorporated herein by reference in their entirety. Aspects of the embodiments can be modified, if necessary to employ concepts of the various patents, applications and publication to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

1. [symbol] H. H. [symbol] [symbol] [symbol] [symbol] [symbol] [symbol] M. Hayka. 1983. 191 c. non-official translation (YAKOVLEV N. N. Chemistry of Motion. The Molecular Basis of Muscle Activity. 1983. Moscow: Nauka [Science]. 191 p.) [in Russian]
2. [symbol] B. B., [symbol] B. B., [symbol] O. A., [symbol] [symbol] [symbol] [symbol] B., [symbol] C. C., [symbol] [symbol] [symbol] B. [symbol] [symbol] [symbol] [symbol] [symbol] [symbol] , 2012, VII, c. 61-66. non-official translation (LIKHVANTSEV V. V., MOROZ V. V., GREBENCHIKOV O. A., GOROKHOVATSKY Yu. I., ZARZHETSKY Yu. V., TIMOSHIN S. S., LEVIKOV D. I., SHAYBAKOVA V. L. Ischemic and Pharmacological Preconditioning. 2012. General Intensive Care Medicine, VII, pp. 61-66) [in Russian]
3. [symbol] [symbol] [symbol] - [symbol] [symbol] [symbol] , [symbol] [symbol] . [symbol] [symbol] [symbol] 2000, [symbol] 7, c. 20-23. non-official translation (VOLKOV N. I. Intermittent Hypoxia: a New Method of Training, Rehabilitation and Therapy. 2000. Physical Education Theory and Practice, No. 7, pp. 20-23) [in Russian]
4. COILA B. The Effects of Too Many Antioxidants. Livestrong.com [online]. Retrieved from https://www.livestrong.com/article/480352-the-effects-of-too-much-antioxidants/THOMAS K. The Dark Side of Antioxidants. BigThink.com [online]. Retrieved from http://bigthink.com/devil-in-the-data/the-dark-side-of-antioxidants
5. HENNEKENS C. H., BURING J. E., MANSON J. E., et al. Lack of Effect of Long-Term Supplementation with Beta-carotene on the Incidence of Malignant Neoplasms and Cardiovascular Disease. New England Journal of Medicine. 1996, Vol. 334, pp. 1145-49
6. HERCBERG S., GALAN P., PREZIOSI P., et al. The SU.VI.MAX Study: a Randomized, Placebo-Controlled Trial of the Health Effects of Antioxidant Vitamins and Minerals. Archives of Internal Medicine. 2004. Vol. 164, pp. 2335-42
7. [symbol] PΦ [symbol] 212 836. [symbol] [symbol] , [symbol] M. H., [symbol] E. A., [symbol] [symbol] , [symbol] A. B., [symbol] A. C. [symbol] , [symbol] [symbol] [symbol] . 1995.01. non-official translation (RU 212836. KAMINSKY Yu. G., KONDRASHOVA M. N., KOSENKO E. A., MAEVSKY E. I., SUCHKOV A. V., ROSENFELD A. S. January 1995. Agent with Actoprotective Activity) [in Russian]
8. [symbol] M. H., [symbol] [symbol] [symbol] [symbol] . [symbol] [symbol] [symbol] [symbol] A H CCCP, 1976, 234 C. non-official translation (KONDRASHOVA, M. N. Therapeutic Effect of Succinic Acid. Collection of Articles. 1976. Pushchino. National Center of Biology Research of the Academy of Sciences of the U.S.S.R. 234 p.) [in Russian]
9. [symbol] [symbol] , [symbol] A. C., [symbol] E. B., [symbol] M. H. [symbol] [symbol] [symbol] [symbol] [symbol] [symbol] [symbol] . [symbol] , 2001. 155 c. non-official translation (MAEVSKY E. I., ROSENFELD A. S., GRISHINA E. V., KONDRASHOVA M. N. Correction of Metabolic Acidosis by Supporting Mitochondrial Functions. 2001. Pushchino. 155 p.) [in Russian]
10. [symbol] P. B., [symbol] O. C., [symbol] [symbol] , [symbol] [symbol] [symbol] [symbol] [symbol] [symbol] [symbol] [symbol] [symbol] [symbol] [symbol] 2016, [symbol] 2, c. 56-59. non-official translation (TAMBOVTSEVA R. V., ZHUMAYEV O. S., VOYTENKO Yu. L., LAPTEV A. I. Influence of Ammonium Succinate on the Aerobic Capacities of Athletes. 2016. Physical Education Theory and Practice, No. 2, pp. 56-59) [in Russian]
11. HAGER H., A Manual on Pharmaceutical and Medical-Chemical Practice, edited and supplemented by Alren, V. K. and Pel, A. V. 1887. Saint Petersburg: K. L. Ricker Publishing House. Volume 1: Ahelomoschus-Capsulae. Ammonium Succinicum solutum [in Russian]
12. [symbol] [symbol] CCCP [symbol] 1090405. [symbol] [symbol] [symbol] [symbol] [symbol] [symbol] “ [symbol] [symbol] ”, 7 Max 1984, [symbol] 17. non-official translation (SU Inventor Certificate No. 1090405. Ammonium Succinate as a Geroprotective Agent. Inventions, Discoveries Bulletin, 7 May 1984. No. 17) [in Russian]
13. [symbol] PΦ [symbol] 2108095. [symbol] A. C., [symbol] B. A., [symbol] M. H., [symbol] [symbol] [symbol] [symbol] [symbol] . 1998. non-official translation (RU 2108095. SARATIKOV A. S., KHAZANOV V. A., KONDRASHOVA M. N., GOLDBERG Yu. M. 1998. Medicinal Agent for the Treatment of Cerebral Ischemia) [in Russian]
14. [symbol] E. A., [symbol] [symbol] , [symbol] [symbol] [symbol] [symbol] [symbol] [symbol] [symbol] [symbol] [symbol] [symbol] [symbol] [symbol] 10- [symbol] [symbol] [symbol] B [symbol] « [symbol] [symbol] [symbol] , [symbol] [symbol] , [symbol] [symbol] », [symbol] , 1997. C. 145-150. non-official translation (MUTUSKINA Ye. A., ZARZHETSKY Yu. V., TRUBINA I. Ye., et al. The Effect of Ammonium Succinate on the Functional, Biochemical and Morphological Indicators of the Recovery of the Central Nervous System in Rats after a 10-Minute Stop of Blood Flow. Succinic Acid in Medicine, the Food Industry and Agriculture. Collection of Articles. 1997. Pushchino. pp. 145-150) [in Russian]
15. [symbol] [symbol] , [symbol] E. A., [symbol] [symbol] [symbol] [symbol] [symbol] [symbol] , [symbol] [symbol] [symbol] [symbol] y [symbol] 10- [symbol] [symbol] [symbol] [symbol] « [symbol] [symbol] [symbol] [symbol] [symbol] [symbol] [symbol] », [symbol] 1997. c. 96-103. non-official translation (ZARZHETSKY Yu. V., MUTUSKINA Ye. A., TRUBINA I. Ye., et al. The Effect of Sodium Succinate on the Functional, Biochemical and Morphological Indicators of the Recovery of the Central Nervous System in Rats after a 10-Minute Stop of Flood Flow. Succinic Acid in Medicine, the Food Industry and Agriculture. Collection of Articles. 1997. Pushchino. pp. 96-103) [in Russian]
16. [symbol] E. A., [symbol] [symbol] , [symbol] [symbol] [symbol] [symbol] [symbol] [symbol] [symbol] [symbol] [symbol] [symbol] [symbol] [symbol] [symbol] B [symbol] « [symbol] [symbol] [symbol] [symbol] [symbol] , [symbol] [symbol] », [symbol] 1997. pp. 151-154. non-official translation (MUTUSKINA Ye. A., ZARZHETSKY Yu. V., TRUBINA I. Ye., et al. The Effect of Succinic Acid on the Post-Resuscitative Pathology of the Central Nervous System and Body as a Whole Based on Data from an Experiment on Rats. Succinic Acid in Medicine, the Food Industry and Agriculture. Collection of Articles. 1997. Pushchino. pp. 151-154) [in Russian]
17. Yantarin-Sport [online]. Retrieved from http://yantarin.ucoz.net/index/jantarin_sport/0-12 [in Russian]
18. BOBKOV Yu. G., VINIGRADOV V. M., KATKOV V. F., LOSEV S. S., SMITNOV A. V. Pharmacological Correction of Tiredness. 1984. Moscow: Meditsina [Medicine]. 208 p. [in Russian]
19. YAKOVLEV N. N. Sport Biochemistry. Fizkultura i Sport [Physical Education and Sport]. 1974. 288 p. [in Russian]
20. HOCHACHKA P. W., SOMERO G. N. Strategies of Biochemical Adaptation. 1973. Saunders College Publishing. (translated from English. 1977. Moscow: Mir Publishing House. 398 p.) [in Russian]
21. NELSON D. L., COX M. M., Lehninger Principles of Biochemistry, Volume 2: Bioenergy and Metabolism. 2012. Freeman/Worth. (translated from English. 2014. Binom Publishing House. 636 p.) [in Russian]
22. [symbol] 1630043. [symbol] E. A., [symbol] , [symbol] H. H., [symbol] Λ. A. 28.02.1994. [symbol] [symbol] [symbol] [symbol] [symbol] . non-official translation (RU 1630043. SELIVANOV Ye. A., SLEPNEVA L. V., ALEKSEYEVA N. N., KHOLODNY G. A. 28 Feb. 1994. Mafusol as a Plasma Replacement Solution for Shock Treatment) [in Russian]
23. [symbol] [symbol] [symbol] M. A. [symbol] [symbol] [symbol] [symbol] [symbol] [symbol] [symbol] [symbol] [symbol] [symbol] [symbol] medline.ru 2012. TOM 13, CT. 82, c. 987-1001. non-official translation (KHANEVICH M. D., GIPAROVICH M. A., SLEPNEVA L. V. History of Konfumin Usage for the Treatment of Hypovolemia and Oncopathology in Adult Patients. medline.ru [online biomedical journal]. 2012. Volume 13, Article 82, pp. 987-1001) [in Russian]
24. [symbol] A. A. [symbol] « [symbol] [symbol] ». [symbol] [symbol] , 2012, 776 c. non-official translation (SARAFANOVA A. A. Encyclopedia of Food Supplements. 2012. Saint Petersburg: Professiya Publishing House. 776 p.) [in Russian]
25. [symbol] A. B., [symbol] E. H., HeMOB M. H., [symbol] , [symbol] [symbol] [symbol] [symbol] [symbol] [symbol] [symbol] [symbol] [symbol] [symbol] [symbol] [symbol] [symbol] [symbol] . 2008. T. 53, [symbol] 6. C. 1025-1032. non-official translation (BEREZHNOV A. V., FEDOTOVA Ye. I., NEMOV M. N., KOKOZ Yu. M., ZINCHENKO V. P., DYNNIK V. V. Destabilization of the Level of Cytosolic Calcium and the Death of Cardiomyocytes in the Presence of Derivatives of Long-Chain Fatty Acids. Biofizika [Biophysics]. 2008. Volume 53, No. 6, pp. 1025-1032) [in Russian]
26. MIRONOVA et al. Biochimica et Biophysica Acta. 2015.1848 (2) pp. 488-495
   BELOSLUDTSEV et al. BBA-Bioenergetics, BBA Biomembranes. 2009.41, pp. 395-401
27. HE W., MLAO F., LIN D., SCHWANDNER R. T., WANG Z., GAO J., CHEN J. L., TLAN H., LING L. Citric Acid Cycle Intermediates as Ligands for Orphan G-Protein-Coupled Receptors. Nature. 2004. Volume 429, pp. 188-193
28. TORMOS K. V., CHANDEL N. S. Inter-Connection between Mitochondria and HIFs. Journal of Cellular and Molecular Medicine. 2010. Volume 14, No. 4, pp. 795-804
29. [symbol] M. H., [symbol] [symbol] [symbol] [symbol] P. H. [symbol] [symbol] [symbol] [symbol] [symbol] [symbol] [symbol] [symbol] B [symbol] [symbol] [symbol] [symbol] . M., 1973, c. 112-129. non-official translation (KONDRASHOVA M. N., MAEVSKY E. I., BABAYAN G. V., SAAKYAN I. R., AKHMEROV R. N. Adaptation to Hypoxia by Switching Metabolism to Succinic Acid Transformations. Mitochondriae. Biochemistry and Ultra-Structure. Collection of Articles. 1973. Moscow, pp. 112-129) [in Russian]
30. LUKIANOVA L. D. Molecular Mechanisms of Tissue Hypoxia and Organism Adaptation. Kyiv, Ukraine: Fiziologichnyi Zhurnal [Journal of Physiology]. 2003.49 (3), pp. 17-35 [in Russian and Ukrainian]
31. [symbol] , [symbol] E. B., [symbol] A. C. [symbol] [symbol] [symbol] [symbol] [symbol] [symbol] - [symbol] [symbol] [symbol] [symbol] [symbol] [symbol] [symbol] 2000. T. 45. [symbol] 3. C. 509-513. non-official translation (MAEVSKY E. I., GRISHINA Ye. V., ROSENFELD A. S., et al. Anaerobic Formation of Succinate and the Facilitation of Its Oxidation as Possible Mechanisms of Cell Adaptation to Oxygen Starvation. Biofizika [Biophysics]. 2000. Volume 45, No. 3, pp. 509-513) [in Russian]
32. VON KORFF R. W. Changes in Metabolic Control Sites of Rabbit Heart Mitochondria. Nature. 1967. Volume 214, pp. 23-26
33. OLSON M. S., VON KORFF R. W. Changes in Endogenous Substrates of Solated Rabbit Heart Mitochondria during Storage. Journal of Biological Chemistry. 1967. Volume 242, No. 2, pp. 325-332
34. [symbol] , [symbol] [symbol] [symbol] [symbol] [symbol] [symbol] [symbol] [symbol] [symbol] [symbol] [symbol] ( [symbol] ). [symbol] 1971. 28 c. non-official translation (MAEVSKY E. I. Influence of Hypoxia and Glutamate on the Responses of the Respiratory Chain of Mitochondria of Certain Organs. Abstract of Doctorate Thesis in Biochemistry. 1971. Sverdlovsk, U.S.S.R. 28 p.) [in Russian]
35. CASCARANO J, ADES I. Z., CONNOR J. D. Hypoxia: a Succinate-Fumarate Electron Shuttle between Peripheral Cells and Lung. Journal of Experimental Zoology. 1976. 198, pp. 149-154
36. NYHAN W. L, BUSCH H. Metabolic Patterns for Succinate-2-C¹⁴ in Tissues of Tumor-Bearing Rats. Cancer Research. November 1958. Volume 18, pp. 1203-1208
37. GILISSEN J., JOURET F., PIROTTE B., HANSON J. Insight into SUCNR1 (GPR91) Structure and Function. Pharmacology and Therapeutics. 2016. Volume 159 pp. 45-56
38. ROTH M., OBAIDAT A., HAGENBUCH B. OATPs, OATs and OCTs: the Organic Anion and Cation Transporters of the SLCO and SLC22A Gene Superfamilies. British Journal of Pharmacology. March 2012. 165(5), pp. 1260-1287
39. [symbol] [symbol] . [symbol] [symbol] [symbol] [symbol] [symbol] [symbol] [symbol] [symbol] [symbol] [symbol] [symbol] [symbol] [symbol] [symbol] [symbol] « [symbol] » M. MTY. 1997. 22 c. non-official translation (YEVTODIYENKO V. Yu. Non-Electrogenic Transport of Hydrogen Ions through a Bilayer Lipid Membrane Induced by Carboxylic Acids with Different Chemical Structures. Abstract of Doctorate Thesis in Biophysics. Moscow: Moscow State University, 1997, 22 p.) [in Russian]
40. LANG T. A. How to Write, Publish, and Present in the Health Sciences. A Guide for Physicians and Laboratory Researchers. American College of Physicians. 2009. 387 p. How to Report Statistics in Medicine (2^{nd} edition). American College of Physicians. 2006. 490 p.
41. SADAGOPAN N., WENLIN Li, ROBERDS S. L., MAJOR T., PRESTON G. M., YING Yu, TONES M. A. Circulating Succinate is Elevated in Rodent Models of Hypertension and Metabolic Disease. American Journal of Hypertension. 2007. Volume 20, pp. 1209-1215
42. GB 1063465 (KAKEN, KAGAKU, KABUSHIKI, KAISHA and TADATAKA HARA) 30 Mar. 1967. Succinic Acid Conformers and Processes for Preparing the Same. (Application No. 12447 made in Japan on 15 Mar. 1963)
43. CHEN T. T., MAEVSKY E. I., UCHITEL M. L. Maintenance of Homeostasis in the Aging Hypothalamus: The Central and Peripheral Roles of Succinate. Frontiers in Endocrinology. Lausanne. 2015. 6:7. 20 p. Published online: 2 Feb. 2015. doi: 10.3389/fendo.2015.00007. PMCID: PMC4313775
44. [symbol] [symbol] [symbol] [symbol] [symbol] Caxaposa [symbol] , [symbol] E. Φ., [symbol] , [symbol] M. H. [symbol] [symbol] [symbol] [symbol] [symbol] [symbol] [symbol] [symbol] [symbol] [symbol] [symbol] 2008. 21:2, c. 298-305. non-official translation (MAEVSKY E. I., PESKOV A. B., UCHITEL M. L., POGORELOV A. G., SAKHAROVA N. Yu., VIKHLYANTSEVA E. F., BOGDANOVA L. A, KONRASHOVA M. N. Mitigating Menopause Symptoms with a Succinate-Based Composition without Hormone-Replacement Therapy. Uspekhi Gerontologii [Advances in Gerontology]. 2008. 21(2), pp. 298-305) [In Russian]
45. KUZNETSOVA I. V., USPENSKAYA Y. B., BORISOVA N. I., ZHUKOVA E. V., BERDNIKOVA N. G., GUSAK Y. K. Effectiveness and Safety of Using a Succinate-Based Dietary Supplement for Women in Menopause. medline.ru [online biomedical journal]. 2016. Vol. 17, Article 3, pp. 24-45
46. MERRY T. L., RISTOW, M. Do Antioxidant Supplements Interfere with Skeletal Muscle Adaptation to Exercise Training? Journal of Physiology. 15 Sep. 2016. 594(18), pp. 5135-5147
47. SAHLIN K., TONKONOGI M., SODERLUND K. Energy Supply and Muscle Fatigue in Humans. Acta Physiologica Scandinavica. 1998. 162(3), pp. 261-266
48. [symbol] A. C., [symbol] [symbol] [symbol] [symbol] [symbol] [symbol] [symbol] [symbol] [symbol] [symbol] [symbol] [symbol] 2004. 4: 39-44. non-official translation (ROSENFELD A. S., MAEVSKY E. I. Stress and Certain Problems with Adaptational Changes during Athletic Workloads. Teoriya i Praktika Fizicheskoi Kultury [Theory and Practice of Physical Education]. 2004. Volume 4, pp. 39-44) [in Russian]
49. [symbol] [symbol] [symbol] [symbol] [symbol] [symbol] [symbol] M.: [symbol] , 2012: 944 C. non-official translation (A Guide to Preclinical Studies of Medicinal Agents. Part 1. 2012. Moscow: Grif & Co. 944 p.) [in Russian]
50. [symbol] B. A. [symbol] [symbol] [symbol] [symbol] [symbol] [symbol] [symbol] [symbol] M. [symbol] [symbol] 2006. 127 c. non-official translation (KISELEV V. A. Perfecting the Athletic Training of Highly Skilled Boxers: an Instruction Manual. 2006. Moscow: Fizicheskaya Kultura [Physical Education]. 127 p.) [in Russian]
51. [symbol] , [symbol] A. A., [symbol] , [symbol] O. B. [symbol] [symbol] [symbol] [symbol] [symbol] [symbol] [symbol] [symbol] [symbol] [symbol] [symbol] 2016. 5, c.3-8. non-official translation (VASILYEV G. F., NOVIKOV A. A., KRUTNIK Ye. Ya., TIUNOVA O. V. Evaluation of Competitive Activity as a Basis for Forecasting the Outcome in Martial Arts Competitions. Vestnik Sportivnoi Nauki [Sports Science Bulletin]. 2016. Volume 5, pp. 3-8) [in Russian]

1. A dosage form for oral administration, comprising a formulation comprising:
about 24%-36% by weight ammonium succinate;
about 4%-16% by weight sodium succinate;
about 4%-12% by weight succinic acid;
about 8%-16% by weight potassium fumarate or sodium fumarate;
about 2%-8% by weight ammonium phosphate; and
about 8%-16% by weight L-carnitine fumarate or a mixture of L-carnitine and sodium fumarate at a 1:1 ratio.

2. The dosage form of claim 1, wherein the formulation comprises 8%-16% potassium fumarate.

3. The dosage form of claim 1 or 2, wherein the formulation comprises 8%-16% L-carnitine fumarate.

4. The dosage form of any one of claims 1-3, wherein sodium succinate is sodium succinate dibase hexahydrate.

5. The dosage form of any one of claims 1-4, wherein one or more components of the formulation is microencapsulated.

6. The dosage form of any one of claims 1-5, wherein the formulation further comprises a pharmaceutical excipient.

7. The dosage form of claim 6, wherein the excipient comprises a filler, a lubricant, an anticaking agent, or any combination thereof

8. The dosage form of claim 7, wherein the formulation comprises filler in an amount of about 10%-20% by weight.

9. The dosage form of claim 7 or 8, wherein the filler comprises microcrystalline cellulose.

10. The dosage form of any one of claims 7-9, wherein formulation comprises lubricant in an amount of about 1%-5% by weight.

11. The dosage form of any one of claims 7-10, wherein the lubricant comprises magnesium stearate.

12. The dosage form of any one of claims 7-11, wherein the formulation comprises anticaking agent in an amount of about 1%-5% by weight.

13. The dosage form of any one of claims 7-12, wherein the anticaking agent comprises silicon dioxide.

14. The dosage form of any one of claims 1-13, wherein the ratios of ammonium succinate, sodium succinate, succinic acid, potassium fumarate or sodium fumarate, ammonium phosphate, and L-carnitine fumarate or a mixture of L-carnitine and sodium fumarate at a 1:1 ratio are about 5:1:1:2:1:2, respectively.

15. The dosage form of any one of claims 1-14, wherein the dosage form is a capsule, tablet, powder, or liquid.

16. The dosage form of any one of claims 1-15, wherein the amount of formulation in the dosage form is about 150 mg 1,700 mg.

17. The dosage form of claim 16, wherein the amount of formulation in the dosage form is about 250 mg 600 mg.

18. The dosage form of claim 16, wherein the amount of formulation in the dosage form is about 300 mg 500 mg.

19. The dosage form of claim 16, wherein the amount of formulation in the dosage form is about 350 mg 400 mg.

20. The dosage form of claim 16, wherein the amount of formulation in the dosage form is about 375 mg.

21. The dosage form of any one of claims 15-20, wherein the dosage form is a capsule and further comprises a capsule shell.

22. The dosage form of claim 21, wherein the capsule shell comprises hydroxypropyl methylcellulose.

23. The dosage form of any one of claims 15-20, wherein the dosage form is a tablet.

24. The dosage form of claim 23, wherein the tablet is coated to 1%-10% weight gain with a film coating.

25. The dosage form of claim 24, wherein the film coating is a water resistant coating.

26. A method of enhancing physical performance or recovery following physical performance in a subject, comprising orally administering an effective amount of the dosage form of any one of claims 1-25 to the subject.

27. The method according to claim 26, wherein the dosage form is administered to the subject at about 3-25 mg/kg.

28. The method according to claim 26, wherein the dosage form is administered to the subject at about 5-20 mg/kg.

29. The method according to claim 26, wherein the dosage form is administered to the subject at about 8-18 mg/kg.

30. The method according to claim 29, wherein the dosage form is administered to the subject about 12-18 mg/kg.

31. The method according to any one of claims 26-30, wherein the dosage forms is administered to the subject about one to three times per day.

32. The method according to any one of claims 26-31, wherein the dosage form is administered to the subject for an initial treatment period of about 2-60 consecutive days.

33. The method according to any one of claims 26-32, wherein the dosage form is administered to the subject for an initial treatment period of about 2-7 consecutive days.

34. The method according to any one of claims 26-32, wherein the dosage form is administered to the subject for an initial treatment period of about 14-28 consecutive days.

35. The method according to any one of claims 26-32, wherein the dosage form is administered to the subject for an initial treatment period of about 30-60 consecutive days.

36. The method according to any one of claims 32-35, wherein following the initial treatment period is a period of about 2-14 consecutive days in which the dosage form is not administered to the subject before resuming treatment with the dosage form.

37. The method according to any one of claims 32-36, wherein following an initial treatment period is a period of about 10-14 consecutive days in which the dosage form is not administered to the subject before resuming treatment with the dosage form.

38. The method according to claim 32, wherein following an initial treatment period of about 60 consecutive days is a period of about 14 consecutive days in which the dosage form is not administered to the subject before resuming treatment with the dosage form.

39. The method according to any one of claims 32-38, wherein the dosage form is administered to the subject with or immediately following a meal.

40. The method according to any one of claims 32-39, wherein the dosage form is administered to the subject about 15-90 minutes before exercise.

41. The method according to any one of claims 32-38, wherein the dosage form is administered to the subject after exercise.

42. The method according to any one of claims 26-41, wherein the subject is human.

43. The method according to any one of claims 26-42, wherein the subject is an adult.

44. The method according to any one of claims 26-43, wherein the subject is an athlete.

45. The method according claim 42, wherein the subject is about 18-80 years old.

46. The method according to claim 45, wherein the subject is about 18-65 years old.

47. The method according to claim 45, wherein the subject is about 18-50 years old.

48. The method according to any one of claims 26-44, wherein the subject is about 18-40 years old.

49. The unit dosage form of any one of claims 1-25 for use in enhancing physical performance or recovery following physical performance in a subject.

50. A dosage form for oral administration, comprising a formulation comprising:
about 54%-76% by weight ammonium succinate;
about 6%-14% by weight sodium succinate; and
about 6%-14% by weight ascorbic acid.

51. The dosage form of any one of claim 50, wherein sodium succinate is sodium succinate dibase hexahydrate.

52. The dosage form of claim 52, wherein one or more components of the formulation is microencapsulated.

53. The dosage form of claim 52, wherein the component that is microencapsulated is ascorbic acid, ammonium succinate, or both.

54. The dosage form of claim 52 or 53, wherein the component is microencapsulated with palm oil, soybean oil, cottonseed oil, sunflower oil, or coconut oil.

55. The dosage form of any one of claims 50-54, wherein the formulation further comprises a pharmaceutical excipient.

56. The dosage form of claim 55, wherein the excipient comprises a lubricant, an anticaking agent, or both.

57. The dosage form of claim 56, wherein the formulation comprises lubricant in an amount of about 1%-5% by weight.

58. The unit dosage form of claim 56 or 57, wherein the lubricant is magnesium stearate.

59. The dosage form of any one of claims 56-58, wherein the formulation comprises anticaking agent in an amount of about 1%-6% by weight.

60. The dosage form of any one of claims 56-59, wherein the anticaking agent is silicon dioxide.

61. The dosage form of any one of claims 50-60, wherein the ratios of ammonium succinate, sodium succinate, and ascorbic acid are at about 10:2:1, respectively.

62. The dosage form of any one of claims 50-61, wherein the dosage form is a capsule, tablet, powder, or liquid.

63. The dosage form of any one of claims 50-62, wherein the amount of formulation in the dosage form is about 150 mg 1,700 mg.

64. The dosage form of claim 63, wherein the amount of formulation in the dosage form is about 250 mg 600 mg.

65. The dosage form of claim 63, wherein the amount of formulation in the dosage form is about 300 mg 500 mg.

66. The dosage form of claim 63, wherein the amount of formulation in the dosage form is about 350 mg 400 mg.

67. The dosage form of claim 63, wherein the amount of formulation in the dosage form is about 360 mg.

68. The dosage form of any one of claims 62-67, wherein the dosage form is a capsule and further comprises a capsule shell.

69. The dosage form of claim 68, wherein the capsule shell comprises hydroxypropyl methylcellulose.

70. The dosage form of claim 62-67, wherein the dosage form is a tablet.

71. The dosage form of claim 70, wherein the tablet is coated to 1%-10% weight gain with a film coating.

72. The dosage form of claim 71, wherein the film coating is a water resistant coating.

73. A method of enhancing physical performance or recovery following physical performance in a subject comprising orally administering an effective amount of a dosage form of any one of claims 50-72 to the subject.

74. The method according to claim 73, wherein the dosage form is administered to the subject at about 3-25 mg/kg.

75. The method according to claim 74, wherein the dosage form is administered to the subject at about 5-20 mg/kg.

76. The method according to claim 74, wherein the dosage form is administered to the subject at about 8-18 mg/kg.

77. The method according to claim 74, wherein the dosage form is administered to the subject at about 12-18 mg/kg.

78. The method according to any one of claims 73-77, wherein the dosage form is administered to the subject about one to about three times per day.

79. The method according to any one of claims 73-78, wherein the dosage form is administered to the subject for an initial treatment period of about 2-60 consecutive days.

80. The method according to claim 79, wherein the dosage form is administered to the subject for an initial treatment period of about 2-7 consecutive days.

81. The method according to claim 79, wherein the dosage form is administered to the subject for an initial treatment period of about 14-28 consecutive days.

82. The method according to claim 79, wherein the dosage form is administered to the subject for an initial treatment period of about 30-60 consecutive days.

83. The method according to any one of claims 79-82, wherein following the initial treatment period is a period of about 2-14 consecutive days in which the dosage form is not administered to the subject before resuming treatment with the dosage form.

84. The method according to any one of claims 79-82, wherein following the initial treatment period is a period of about 10-14 days in which the dosage form is not administered to the subject before resuming treatment with the dosage form.

85. The method according to claim 79, wherein following an initial treatment period of about 60 consecutive days is a period of about 14 consecutive days in which the dosage form is not administered to the subject before resuming treatment with the dosage form.

86. The method according to any one of claims 73-85, wherein the dosage form is administered to the subject with or immediately following a meal.

87. The method according to any one of claims 73-86, wherein the dosage form is administered to the subject about 15-90 minutes before exercise.

88. The method according to claim 87, further comprising administering to the subject the dosage form of any one of claims 1-25 after exercise.

89. The method according to claim 88, wherein an effective amount of the dosage form of any one of claims 1-25 is administered to the subject at least two hours after the dosage form of any one of claims 50-72 is administered.

90. The method of any one of claims 73-89, wherein the subject is human.

91. The method of any one of claims 73-90, wherein the subject is an adult.

92. The method according to of claim 90, wherein the subject is an athlete.

93. The method according to claim 90, wherein the subject is about 18-65 years old.

94. The method according to claim 90, wherein the subject is about 18-45 years old.

95. A dosage form for oral administration, comprising a formulation comprising:
about 36%-48% by weight ammonium succinate;
about 7%-14% by weight sodium succinate;
about 4%-9% by weight succinic acid;
about 4%-7% by weight potassium fumarate or sodium fumarate;
about 2%-4% by weight ammonium phosphate;
about 8%-14% by weight L-carnitine fumarate or a mixture of L-carnitine and sodium fumarate at a 1:1 ratio;
about 1%-3% by weight antioxidant;
about 4%-10% by weight dihydroquercetin;
about 4%-10% by weight α-tocopherol; and
about 4%-14% by weight ascorbic acid.

96. The dosage form of claim 95, wherein the antioxidant is curcumin or lycopene.

97. The dosage form of claim 96, wherein the antioxidant is curcumin.

98. The dosage form of claim 96, wherein the antioxidant is curcumin, demethoxycurcumin, bisdemethoxycurcumin, or a combination thereof.

99. The dosage form of claim 96, wherein the antioxidant is lycopene.

100. The dosage form of claim 99, wherein the lycopene comprises lycopene-13-cis-isomer.

101. The dosage form of any one of claims 95-100, wherein sodium succinate is sodium succinate dibase hexahydrate.

102. The dosage form of any one of claims 95-101, wherein one or more components of the formulation is microencapsulated.

103. The dosage form of claim 102, wherein the component that is microencapsulated is α-tocopherol.

104. The dosage form of claim 102 or 103, wherein the component that is microencapsulated is ascorbic acid.

105. The dosage form of any one of claims 102-104, wherein the component that is microencapsulated is curcumin.

106. The dosage form of claim 102, wherein α-tocopherol, ascorbic acid, and curcumin are microencapsulated together or separately.

107. The dosage form of any one of claims 102-106, wherein the component is microencapsulated with palm oil, soybean oil, cottonseed oil, sunflower oil, or coconut oil.

108. The dosage form of any one of claims 95-107, wherein the formulation further comprises a pharmaceutical excipient.

109. The dosage form of claim 108, wherein the excipient comprises a lubricant, an anticaking agent, or both.

110. The dosage form of claim 109, wherein the formulation comprises lubricant in an amount of about 0.1%-5% by weight.

111. The dosage form of claim 109 or 110, wherein the lubricant is magnesium stearate.

112. The dosage form of any one of claims 109-111, wherein the formulation comprises anticaking agent in an amount of about 2-4% by weight.

113. The dosage form of any one of claims 109-112, wherein the anticaking agent is silicon dioxide.

114. The dosage form of any one of claims 95-113, wherein the ratios of ammonium succinate, sodium succinate, succinic acid, potassium fumarate or sodium fumarate, ammonium phosphate, L-carnitine fumarate or a mixture of L-carnitine and sodium fumarate in a 1:1 ratio, antioxidant, dihydroquercetin, α-tocopherol, and ascorbic acid are 10:2:1.13:1.33:0.53:2.33:0.4:1.13:1.13:1, respectively.

115. The dosage form of any one of claims 95-114, wherein the dosage form is a capsule, tablet, powder, or liquid.

116. The dosage form of any one of claims 95-115, wherein the amount of formulation in the dosage form is about 150 mg 1,700 mg.

117. The dosage form of claim 116, wherein the amount of formulation in the dosage form is about 250 mg 600 mg.

118. The dosage form of claim 116, wherein amount of formulation in the dosage form is about 300 mg 500 mg.

119. The dosage form of claim 116, wherein the amount of formulation in the dosage form is about 350 mg 400 mg.

120. The dosage form claim 116, wherein the amount of formulation in the dosage form is about 350 mg.

121. The dosage form of any one of claims 115-120, wherein the dosage form is a capsule and further comprises a capsule shell.

122. The dosage form of claim 121, wherein the capsule shell comprises hydroxypropyl methylcellulose.

123. The dosage form of any one of claims 115-120, wherein the dosage form is a tablet.

124. The dosage form of claim 123, wherein the table is coated to 1-10% weight gain with a film coating.

125. The dosage form of claim 124, wherein the film coating is a water resistant coating.

126. A method for enhancing physical performance, recovery following physical performance in a subject comprising orally administering an effective amount of a dosage form of any one of claims 95-125 to the subject.

127. The method according to claim 126, wherein the dosage form is administered to the subject at about 3-25 mg/kg.

128. The method according to claim 127, wherein the dosage form is administered to the subject at about 5-20 mg/kg.

129. The method according to claim 127, wherein the dosage form is administered to the subject at about 8-18 mg/kg.

130. The method according to claim 127, wherein the dosage form is administered to the subject at about 12-18 mg/kg.

131. The method according to any one of claims 126-130, wherein the dosage forms is administered to the subject about one to about three times per day.

132. The method according to any one of claims 126-131, wherein the dosage form is administered to the subject for an initial treatment period of about 2-60 consecutive days.

133. The method according to claim 132, wherein the dosage form is administered to the subject for an initial treatment period of about 2-7 consecutive days.

134. The method according to claim 132, wherein the dosage form is administered to the subject for an initial treatment period of about 14-28 consecutive days.

135. The method according to claim 132, wherein the dosage form is administered to the subject for an initial treatment period of about 30-60 consecutive days.

136. The method according to any one of claims 132-135, wherein the initial treatment period is followed by a period of about 2-14 consecutive days in which the dosage form is not administered to the subject before resuming treatment with the dosage form.

137. The method according to any one of claims 132-135, wherein the initial treatment period is followed by a period of about 10-14 consecutive days in which the dosage form is not administered to the subject before resuming treatment with the dosage form.

138. The method according to claim 132, wherein the dosage form is administered for an initial treatment period of about 60 consecutive days followed by a period of about 14 consecutive days in which the dosage form is not administered to the subject before resuming treatment with the dosage form.

139. The method according to any one of claims 126-138, wherein the dosage form is administered to the subject with or immediately following a meal.

140. The method according to any one of claims 126-139, wherein the dosage form is administered to the subject after exercise.

141. The method according to claim 140, further comprising administering to the subject an effective amount of the dosage form of any one of claims 1-25 about 15-90 minutes before exercise.

142. The method according to claim 141, wherein the dosage form of any one of claims 95-125 is administered to the subject at least two hours after the dosage form of any one of claims 1-25 is administered.

143. The method according to any one of claims 126-142, wherein the subject is an active adult.

144. The method according to any one of claims 126-143, wherein the subject is human.

145. The method according to claim 144, wherein the subject is about 50-80 years old.

146. The method according to 145, wherein the subject is about 50-75 years old.
